(19) United States
(12) Patent Application Publication
(10) Pub. No.: US 2025/0279159 A1
(43) Pub. Date: Sep. 4, 2025

(54) SYSTEM AND METHOD FOR DETERMINING AND DEVELOPING MONOCLONAL ANTIBODIES AS BIOMARKERS FOR DIAGNOSTIC TARGETS AND THERAPEUTIC APPLICATIONS

(71) Applicant: Steven Heron, Berwyn, PA (US)

(72) Inventor: Steven Heron, Berwyn, PA (US)

(21) Appl. No.: 19/065,360

(22) Filed: Feb. 27, 2025

Related U.S. Application Data
(60) Provisional application No. 63/560,460, filed on Mar. 1, 2024.

(51) Int. Cl. G16B 15/30; G16B 15/20; G16B 40/20

(52) U.S. Cl. CPC G16B 15/30; G16B 15/20; G16B 40/20

(57) ABSTRACT

Disclosed is a method for developing antibody-based treatments, and HLA- or auto-antibody-based treatments in particular. The method may include determining or receiving a target HLA- or auto-antibody, where the target HLA- or auto-antibody has been identified using the disclosed NETRAD (neoepitope transplant rejection and autoimmune disease) algorithm. The method may include utilizing that antibody to build the desired treatment. The method may include removing a functional portion of target HLA- or auto-antibody. The method may include attaching one or more functional molecules (such as a nanoparticle, a drug, a biological functional moiety, or a dye) to the modified antibody. The method may include development of computer software for epitope analysis of (anti-) HLA antibodies for risk assessment of organ transplant rejection.

### CROSS-REFERENCE TO RELATED APPLICATIONS

The present application claims priority to U.S. Provisional Patent Application No. 63/560,460, filed Mar. 1, 2024, the contents of which are incorporated by reference herein in its entirety.

### REFERENCE TO A SEQUENCE LISTING

The present application is being filed along with a Sequence Listing in ST.26 XML format. The Sequence Listing is provided as a file titled “HERON-001_ST26.XML” created 19 Jun. 2023 and is 30,863 bytes in size. The Sequence Listing information in the ST.26 XML format is incorporated herein by reference in its entirety.

### TECHNICAL FIELD

The present disclosure is drawn to techniques for identifying and developing polyspecific monoclonal anti-HLA and autoimmune antibodies as biomarkers of viral coinfection for diagnostic targets, as diagnostic indicators of transplant rejection and as therapeutic treatments for organ transplant rejection and HLA-associated chronic diseases (multiple sclerosis, narcolepsy, type I diabetes, psoriasis, celiac disease, and Alzheimer's disease to name a few).

### BACKGROUND

Human leukocyte antigen (HLA) protein antigens distinguish an individual's immune recognition repertoire by nature of their unique polymorphisms. Over 35,000 distinct HLA allelic variants have been catalogued. The hypervariability of amino acid residue mutations, when compared among HLA gene locus sequences, are the key to characterizing an individual's inherited HLA phenotype. By contrast, combinations of distinct residue mutations may be shared among HLA allele subsets to provide the basis for cross-reactive (public) epitopes. This complex variability is distributed over multiple HLA gene loci. Transplant diagnostic tests rely on this complexity to facilitate identification of antibody specificities that are important to risk assessment for antibody-mediated rejection.

Problems in the scientific literature gradually surfaced while attempting to characterize antibody specificities that target unique HLA antigenic epitopes. These problems persist to this day. HLA antigenic epitopes were originally characterized by mapping serum reactivity to short amino acid residue sequence configurations designated as functional eplets that identify a unique HLA epitope specificity. The complementary binding surface of an antibody, known as a paratope, binds to its targeted epitope by encompassing the functional eplet. Presently, currently recognized HLA epitopes listed in at least one prevailing database (www.epregistry.com.br/) recognize most HLA functional eplets as being characterized by 1-2 residues located at hypervariable sites.

However, 1-2 residues alone are inadequate to convey sufficient antibody specificity. The current practice of utilizing such a small number of amino acids to delineate specificity does not permit accurate distinction between an anti-HLA antibody, an autoimmune antibody or an antibody targeting a pathogen. In fact, most antibody paratopes are comprised of 15-25 contact residues to achieve functional specificity.

### BRIEF SUMMARY

In various aspects, a method for developing a diagnostic interpretation tool and antibody-based treatment may be provided. The method may include identifying coinfecting or superinfecting viral agents in organ transplant or autoimmune patients, prophylactically (e.g., pre-transplant or pre-disease) that are at risk of developing rejection (e.g., alloimmune/non-self in the case of transplantation; autoimmune/self in the case of autoimmune disease) or post-disease onset. The method may include identifying the concurrence of deleterious HLA donor-specific anti-HLA antibodies (DSA) (HLADSA) or autoantibodies with non-DSA or anti-anti-idiotype antibodies or exosomes containing modified viral components to determine the exacerbation of allograft rejection and/or autoimmune disease. The method may include identifying a target human leukocyte antigen (HLA)-antibody specificity or auto-antibody. The target HLA-antibody specificity or auto-antibody may have been identified via several steps. The steps may include obtaining a plurality of HLA, autoantigen, and viral protein sequences. The steps may include creating at least one in silico library of a plurality of enzyme-digested viral peptides. The steps may include assessing each enzyme-digested viral peptide for (i) HLA or autoantigen homology and specificity, and (ii) antibody accessibility to one or more clinically relevant HLA or autoantigen binding sites. The steps may include assigning a relative surface-accessibility score to HLA or autoantigen viral peptide motifs depending on their location relative to an ectodomain, specifically for accessibility to antibody binding. The method may include forming a modified antibody by removing a functional portion of target HLA- or auto-antibody. The method may include forming a target antibody by attaching one or more functional molecules to the modified antibody.

The HLA, autoantigen, and viral protein sequences may be obtained from one or more databases. Each enzyme-digested viral peptide may be an N-lysozyme digested viral peptide. Assessing each enzyme-digested viral peptide for HLA or autoantigen homology and specificity may include utilizing the Smith-Waterman algorithm with bidirectionality.

The method may include mapping at least six enzyme-digested viral peptides that impute gapped homology to HLA-specific or autoantigen-specific amino acid residues on a respective crystal structure, wherein a minimum of one and maximum of five of the six enzyme-digested viral peptides must be derived from Epstein-Barr virus (EBV) envelope glycoprotein gp350 or gB.

The method may include obtaining HLA or autoantigen crystal structures from one or more databases and assessing the HLA or autoantigen crystal structures for antibody accessibility to an epitope.

The identifying of homologous peptide sequences shared between viral envelope proteins and HLA antigens or autoantigens may include identifying viral-derived HLA or autoimmune epitopes displaying at least 7 antibody-accessible residues.

The method may include identifying, on viral envelope protein sequences and individual peptide sequences, independently, a post translational modification (PTM) that is capable of influencing generation of a neoepitope. The neoepitope may be determined by the presence of at least one N-glycan within individual peptide sequences and at least one small ubiquitin-like modifier (SUMO) within the viral envelope protein.

The method may include determining a motif to be a clinically relevant HLA or autoantigen binding site, for HLA only when the motif is unimpeded by peptides presented in an HLA cleft. The method may include mapping a surface-accessible HLA-homologous or autoantigen-homologous viral peptide to a respective HLA or autoantigen crystal structure to determine whether an antibody specificity, comprised of at least 7 amino acids including a functional eplet, could be encompassed inside a 15-angstrom radius.

The functional portion of the HLA- or auto-antibody may include the fragment crystallizable (Fc) region. The functional molecule may include a dye that is conjugated or attached to the modified antibody. The functional molecule may be a chimeric antigen receptor (CAR) that includes a transmembrane domain and a signaling domain. The CAR single chain variable fragment (scFv) may incorporate the identified polyspecific anti-microbial antibody. The functional molecule may be a nanoparticle that includes one or more small interfering RNA (siRNA) molecules.

The identifying of homologous peptide sequences shared between viral envelope proteins and HLA antigens or autoantigens may include identifying at least one coinfection or superinfection of a cell by two distinct viruses.

The method may include introducing a plurality of target antibodies as disclosed herein (which may, e.g., be coupled to a dye) into a subject and allowing the target antibodies to bind to a cell containing a pathogenic virus pair. The method may include excising or treating cells at a location of the target antibodies bound to the pathogenic virus pair in the subject.

In various aspects, a method for estimating the risk for antibody-mediated rejection of cells, tissue or organs based on information relating to both a transplant donor and a transplant recipient. The method may include determining donor- and recipient-exposures to HLA-homologous viral components resulting from prior exposures to viral infections, bacterial infections, blood transfusion, prior transplantations, pregnancies, metabolic stress, or vaccinations, that may function as an HLA or autoreacting sensitization event.

The method may include determining the likelihood of cross-reactivity and antibody mediated rejection to occur if transplantation were to proceed, via several steps. The steps may include combining viral envelope protein sequences into a viral peptide library. The steps may include creating at least one in silico library of a plurality of enzyme-digested viral peptides, generating viral peptide sub-sequences. The steps may include assessing each enzyme-digested viral peptide sub-sequence for (i) HLA- or autoantigen-homology and -specificity, and (ii) antibody accessibility to one or more clinically relevant HLA or autoantigen binding sites. The method may include scoring the viral peptide sub-sequences by assigning a relative surface-accessibility score to HLA or autoantigen viral peptide motifs depending on their location relative to an ectodomain, specifically for accessibility to antibody binding. The method may include assessing the location of each motif of the corresponding HLA crystal structure to determine α-helix surface-accessibility. The method may include determining motifs unimpeded by peptides presented in the HLA cleft. The method may include mapping surface-accessible, HLA-homologous viral peptides to respective HLA crystal structures. The method may include identifying post-translational modifications, specifically N-glycan and SUMO sequons, that may influence the generation of neoepitopes on viral envelope protein sequences and on individual peptide sequences. The method may include identifying homologous peptide sequences shared between viral envelope proteins and HLA antigens or autoantigens. The method may include estimating the risk for antibody-mediated rejection of a transplant donor's cells, tissue or organs in a transplant recipient's body.

The method may include receiving and/or obtaining protein sequences for a donor's and a transplant recipient's HLA-type. The method may include receiving and/or obtaining a plurality of HLA, autoantigen, and viral envelope protein sequences known to be involved in a patient's prior exposures to viral infections, bacterial infections, blood transfusion, prior transplantations, pregnancies, metabolic stress, or vaccinations.

### BRIEF DESCRIPTION OF THE DRAWINGS

FIG. 1 is a flowchart of the NETRAD algorithm.

FIG. 2 is a representative image of an HLA-specific epitope from viral peptides superimposed onto a crystal structure.

FIGS. 3A and 3B are images of HLA protein (PDB 5w69) crystal structure from two different rotation angles as a visual aid for NETRAD ectodomain-relative peptide scoring locations (ERP=0-5) used to assess antibody accessibility.

FIGS. 4A and 4B are amino acid sequences and PTMs for Epstein Barr virus glycoprotein 350 (YP_401667.1 1986 USA) (FIG. 3A) and SARS-CoV-2 spike protein (OPDB: 7LSS_C/Feb 2021 B.1.1.7 and B.1.351) (FIG. 3B). Verified N-glycan consensus sequons (shown with a dashed box) are emphasized by sequence motif NxS/T in forward or reverse order, where ‘x’=any amino acid and no proline residue is immediately adjacent to, or part of, the motif Unverified glycan sequons are highlighted in bold. EBV gp350 remained unique among viral attachment proteins until 2019, exhibiting the highest number of N-glycan chains, fourteen that are known. The SARS-CoV-2 spike protein surpassed that number with twenty-two. Predicted SUMO2/3 sequon motifs (shown highlighted in black with white underlying text) are highlighted according to the consensus sequence motif Kx(E/D) (where “x” is any amino acid), or alternative SUMO interacting motifs (i.e., LLIV, VSVI, LTVL, etc.), with requisite flanking acidic residues highlighted in gray. PTM motifs can function in forward or reverse order. Protein modification, including the removal of N-glycans (de-glycosylation) and covalent addition of SUMO, predicts complete alteration of gp350 and spike antigenicity, rendering them unrecognizable protein entities by conventional antibody assays.

FIG. 5 is a chart showing a derivation of HLA-A*02:01 DSA (epitope 62GE in Tables 2 and 3) from EBV and CMV viral peptide motifs superimposed upon the HLA sequences of a transplanted patient and organ donor pair, where DSA=reference sequence.

FIG. 6 is an annotated image showing a comparison of idiotype model (left) vs NETRAD neoantigen model (right).

FIG. 7 is an annotated image showing how Anti-HLA-B*57:01 (epitope 62GRN) can behave like an anti-anti-idiotype non-DSA antibody.

### DETAILED DESCRIPTION

Understanding the foundations of HLA antigenicity and immunogenicity are vital to assessing the risk of anti-HLA antibodies among transplant patients. The complexities of identifying and characterizing these antibodies influence donor selection as well as desensitization and immunosuppression protocols that are designed to extend allograft survival. The mechanism(s) which drives the generation of these antibodies dictates the efficacy of diagnostic risk assessment, desensitization and treatment of antibody-mediated allograft rejection. Athorough investigation of the foundations of HLA antigenicity and immunogenicity in both historical and recent literature yielded surprisingly unexpected results: while HLA proteins are definitively antigenic targets of the immune system, we discovered that the basis on which mismatched HLA epitopes were imputed to be immunogenic may be unreliable.

From a historical standpoint, the immunogenic substance generating alloantibodies was originally isolated from regional lymph nodes. In contrast to HLA, this proteinaceous substance was first recovered from the cell nucleus, challenging mainstream theory that imputes mismatched HLA proteins to be immunogenic. When recovered as ‘soluble HLA antigen,’ the heterogeneous substance was contaminated with ceruloplasmin, a protein required for iron and copper homeostasis during hypoxia as well as the production of defective viral particles. In addition, protein contaminants having a higher molecular weight than HLA were present. Upon isolation, “soluble HLA antigen” could not be distinguished between HLA Class I versus the neonatal Fc receptor (FcRn), this detail was not identified until 10 years later. HLA Class I proteins and the FcRn share nearly identical molecular weight and β-2-microglobulin (β2M), which may have resulted in the immunogenic substance being misidentified as HLA Class I. Amino acid sequencing was not performed to confirm the identity of the purified component substance. HLA Class I was assumed to be the purified immunogenic component due to the identification of β2M. However, the source of the immunogenic substance could not be distinguished from viral components, extracellular vesicles (exosomes), or interfering particles that are currently observed in anti-HLA antibody assays. Moreover, identification of HLA as the immunogenic substance cannot fully explain why a high frequency of mismatched donor HLA antigens fail to consistently exhibit immunodominance and high affinity maturation when bound by cross-reactive DSA. Notably, “soluble HLA antigen” inhibits anti-HLA antibody detection in the single antigen bead assay, indicating higher antibody affinity to a distinct entity.

The possibility that viral homology to HLA may have been misinterpreted as “traditional HLA sensitization events” based on evidence that viral particles can transfer from donor to patient during transfusion and transplantation (or from mother to fetus during pregnancy, respectively), concurrent with the donor's HLA could not be ruled out. Influenza viral RNA has been discovered in symptomless blood donors by using more sensitive next generation sequencing techniques and was shown to be passively transferred from mother to child via the placenta. EBV has also been identified in placental tissue. Increasing evidence supports the finding that viruses utilize the exosomal pathway as an immune evasion strategy or alternate propagation method. This evidence supports the hypothesis that exposure to HLA-homologous viral components via blood transfusion, transplantation or pregnancy could indeed function as a “traditional HLA sensitization event” and legitimizes the NETRAD approach. After uncovering that current research relies on early studies' imputing immunogenicity upon HLA, and that this assumption may be unreliable, the inventor searched for viral sources as a stimulus of anti-HLA antibodies.

Since anti-HLA antibodies more frequently could not be accounted for during the course of (or immediately following) a specific viral infection, it was proposed that they could be influenced by multiple viral components that might be reactivated by metabolic changes, including cell stressors such as IRI. It occurred to the inventor that the jumbling of immunogenic components from two or more distinct viruses within the same lymphoid germinal center might explain the generation of an anti-HLA antibody. A scenario of this nature would be consistent with polyreactive antibodies exhibiting relatively lower affinity. It was hypothesized that polyreactive anti-viral antibodies might cross-react with HLA-homologous subsequences and account for the reactivity patterns observed among anti-HLA antibodies.

Various deficiencies arising from use of conventional techniques can be overcome using the disclosed approach.

One aspect of the disclosed technique utilizes an algorithm, termed NETRAD (Neoepitope Transplant Rejection and Autoimmune Disease).

Consistent patterns of EBV coinfection/superinfection may first be observed, including either coinfection/superinfection with a “distinct” microbe (especially a virus) or upon exposure to the “distinct” microbe through vaccination, followed by and that correlate with the development of specific allo- or autoantibodies.

For identification of homology to allo-/auto-epitopes, target human proteins and suspected viral envelope protein sequences can be obtained from IMGT-HLA (www.ebi.ac.uk/ipd/imgt/hla/(2024)/or NCBI (www.ncbi.nlm.nih.gov/(2024)) websites, respectively. Major immunogenic viral envelope attachment proteins are considered likely candidates due to their extent of glycosylation and documented immunogenicity.

In silico N-lysozyme digested viral peptide libraries are created from viral attachment proteins by submitting the amino acid sequence into EXPASY PeptideMass: (web.expasy.org/peptide_mass (2024)/). Peptides are listed and identified by mass.

In silico digested N-Lysozyme-generated viral peptides are assessed for homology and specificity by utilizing the Smith-Waterman algorithm (www.ebi.ac.uk/Tools/psa/emboss_water (2024)/). Peptides are assessed for homology by Smith-Waterman in forward and reverse directions, independently.

Each peptide may be listed in, e.g., a spreadsheet. Homology may be identified using standard amino acid nomenclature. Gapped amino acid positions are listed as “x” or spacers “-” “x” indicates an identical number of gaps when comparing HLA vs viral sequences whereas “-” indicates a gap that is offset between the HLA and corresponding viral sequence.

Peptides are identified by Expasy PeptideMass identifier in the first column of the spreadsheet, followed by an alphabetic indicator of peptide sequence orientation (F=forward, R=reverse). Unique spreadsheet columns indicate the virus envelope protein of origin, number of N-glycan sequons present on the original viral peptide sequence, homology derived from Smith-Waterman analysis of the correlate viral peptide, target protein start residue, an ectodomain relative peptide (ERP) scored location (expounded below) indicating the relative location of the peptide sequence with regard to surface accessibility (e.g., 0-5), and whether the peptide sequence is specific for the target protein (Yes/No).

Each peptide may be mapped to the correlate amino acid residues on the respective targeted protein crystal structure using appropriate databanks and molecular modeling software (e.g., Research Collaboratory for Structural Bioinformatics Protein Data Bank/PDB, Molsoft ICM, YASARA).

Peptide locations may be scored (ERP) according to their position and accessibility to extracellular binding by elements of the immune response/ligand relative to the cell membrane as with an example as follows: 1=membrane-distal; 2=stalk region, 3=membrane-proximal; 4=membrane abutment; 0=cryptic locations lacking antibody-accessible residues (i.e., peptide cleft, buried or transmembrane residues). Locations with an ERP score of “1-3” are positioned for optimal antibody-binding access in the extracellular space. Scores of “0” or “4” may be considered as cryptic locations.

ERP-scored homologous viral peptide motifs may then be sorted in a spreadsheet according to cross-referenced amino acid sequence position of the target protein.

Combinations of viral peptides are mapped to crystal structures to determine whether a clinically relevant target protein-specific epitope can be demarcated. Such an epitope must be encompassed within a 15-angstrom radius and be unencumbered by quaternary structures and post-translational modifications.

Post-translational modification sequons, specifically for Small Ubiquitin-like Molecules (SUMO) and N-glycans and that might influence the generation of neoepitopes under cell stress conditions (e.g., ischemia/reperfusion injury) may be identified for each viral envelope protein and each viral peptide sequence, respectively.

The epitope of a targeted protein can typically be accommodated by 6 homologous viral peptides (for which both forward and reverse direction sequence matching must be considered)

NETRAD identifies HLA-homologous, gapped viral amino acid sequence motifs (e.g., KAxSxT, where capital letters represent homologous amino acids and “x” represents residues that differ between HLA and viral sequences).

One of the 6 homologous viral peptides must be derived from Epstein Barr virus (gp350 or gB) and at least one from microbe #2 (e.g., superinfecting virus); with the exception that the combination of gp350 and gB peptides may complete an epitope configuration. One of the 6 viral peptides must contain an N-glycan sequon. Both viral proteins must contain a SUMO sequon.

Peptides are mapped to the corresponding target protein crystal structure and sorted by ERP score to determine whether an epitope can be generated that meets the criteria to bind to a T cell receptor and/or B cell receptor (immunoglobulin/antibody).

The algorithm enables the recognition of antibodies that behave as though they were “anti-anti-idiotypes”, meaning an antibody that can compete with another antibody (allogeneic or autoimmune) for binding to the same defective virus-like particle or exosome containing the respective viral antigen harboring target homology. The identification of these “anti-anti-idiotypes” is expected to promote exacerbation of pathology and can be utilized to assess diagnostic risk of transplant rejection and severity of autoimmune disease and to identify or modify therapeutic treatment regimens for each, respectively.

To ascertain the specificity of these antibodies for use in diagnostic risk assessment and/or therapeutic treatment, training and validation of the algorithm must be performed to determine the accuracy of an antibody specificity and its effective titer.

It is envisioned that ELISA can be used to ascertain monoclonal allo-/auto-antibody specificity to viral components after obtaining relevant viral proteins. This can be accomplished by coating high binding microtiter plates with relevant viral envelope protein (1-3 ug/mL) and/or individual peptides derived from said protein in a high pH buffer. After which bound proteins will be treated with PNGase or N-lysozyme. Untreated viral envelope proteins and/or non-specific proteins can be used as controls. Detergent (i.e., sodium dodecyl sulfate) may be used to stop the reaction followed by wash and removal (to remove unused enzyme). Blocking buffer (i.e., 3% bovine serum albumin) is added to reduce non-specific binding followed by wash and removal. Monoclonal antibody with suspected reactivity to viral homology is added and incubated to allow specific binding; titrate as appropriate. Include non-specific control monoclonal antibodies. Follow incubation with washing and removal. Incubate with appropriate immunoglobulin class secondary antibody conjugated to substrate (i.e., CDP-STAR from Perkin-Elmer). Read and obtain signal with appropriate instrumentation (i.e., Envision). Comparison of N-lysozyme/PNGase-treated vs non-treated wells should demonstrate an expected increase in signal-to-noise ratio (S/N) for treated viral proteins incubated with correlate HLA-specific monoclonal antibody. Control antibody (non-HLA specific) is expected to demonstrate no increase in S/N or a comparable level to that of the non-treated/non-specific wells.

It is envisioned that “soluble HLA or autoimmune antigen” (sAg), also referred to as serum interfering factors/defective virus-like particles/exosomes, are observed in HLA single antigen bead array and other testing methods and are suspected to be post-translationally modified with SUMO. To confirm suspected PTM-VLP, patient serum can be interrogated for SUMOylated viral proteins. sAg can be captured using an elution column/beads bound with anti-HLA or autoimmune antibody, then eluted from the column using SDS/glycine. Protein can then be captured using ELISA coated with anti-SUMO-2/3, conjugated to the plate surface. Deconjugation of SUMO can be done with SENP3/SENP7. Then the protein can be analyzed with mass spec/3D electrophoresis/protein sequencing.

Confirmation of the NETRAD model may involve development of a humanized mouse model (infect mice with EBV/MHV-68 plus coinfecting virus-Charles River) and/or cell culture to mimic conditions of ischemia/reperfusion injury (IRI): stress conditions (e.g., cigarette smoke, hypothermia to mimic IRI, cobalt chloride, hypoxia chamber) suspected to give rise to anti-HLA/auto-antibodies. A mouse/cell line with a specific HLA typing that is expected to give rise to a particular non-self allo-/auto-antibody is infected/vaccinated with EBV/MHV-68 plus a distinct superinfecting virus expected to produce an antibody. Animal/cell line is subjected to stress environment to upregulate genes associated with production of SUMO2/3 and glycolysis. As antibodies are generated from semi-random V-D-J gene rearrangements, antibodies generated from individual B cells/plasma cells must be screened and characterized for their ability to produce antibody targeting the suspected allogeneic/autoimmune protein as well as cross react with the suspected viral protein/peptide target. Thus, a method is envisioned where a mouse/cell line with a desired/predetermined HLA typing may be infected or vaccinated with EBV and/or MHV-68 as well as an additional infecting virus expected to produce an antibody. The mouse/cell line may then be stressed to upregulate genes associated with production of SUMO2/3 and glycolysis. Antibodies generated from individual B cells/plasma cells may then be screened and characterized as appropriate.

In certain aspects, this algorithm can be incorporated into software designed for determining the likelihood of generating anti-HLA and/or autoimmune antibodies to improve diagnostic and treatment applications. For example, for predicting more accurately the likelihood of a successful outcome of a cell, tissue or organ transplantation, for predicting the type and strength of immunosuppressive and other post-transplantation therapies, for reducing negative side effects for transplant recipient patients, and for increasing the availability of organs to the general patient population via extending graft survival. In addition, the algorithm can be used to engineer monoclonal antibodies that are polyspecific as therapeutic biomarkers for treating many different diseases, ranging from chronic autoimmune diseases including multiple sclerosis, type I diabetes, psoriasis, narcolepsy and celiac disease to Alzheimer's disease and various cancers. It is sort of like a “magic-bullet” approach that is patient-specific.

The disclosed technique utilizes the algorithm to identify specific immune responses which can take place during disease processes based on the patient's prior exposure to viral superinfection, coinfection and other types of infections. The present invention describes how a combination of viral coinfections and metabolic stress-induced molecular modifications can provide an immunogenic stimulus for antibody-mediated organ transplant rejection (AMR) or the onset of chronic autoimmune disease. These specific immune responses are HLA-specific. HLA, human white blood cell proteins that operate to determine self-versus non-self-recognition (aka foreign or pathogenic entities including viruses), are inherited and associated with more diseases than any other product (protein) of the human genome. In the realm of transplantation science, antibodies are frequently made by organ transplant recipients to target the foreign HLA of the donor organ and destroy (reject) it. In the realm of autoimmune disease, the patient develops antibodies that attack self-tissues and begin to destroy (reject) one's own body.

Conventional approaches are based on the theory (in the case of transplant) that HLA antibodies develop as a response to the foreign HLA encountered upon exposure to blood transfusions, pregnancy, or the transplanted organ itself. Alternatively, in the case of autoimmune disease, scientists have speculated that autoimmune antibodies develop in response to tissue damage and exposure to cellular contents that are normally not seen by the immune system.

The present disclosure rejects these theories. The present disclosure provides, inter alia, a common mechanism for both autoimmunity and transplant rejection being caused by 3 main elements: 1) Stress (including but not limited to oxygen deprivation that is associated with transfusion/pregnancy/transplant) causes changes in our bodies that can reactivate “sleeping” viruses and virus components, 2) specific reactivation of elements of the Epstein Barr virus (EBV), including the envelope glycoproteins gp350 and gB, and 3) reactivation of envelope proteins of another virus which co-infects the same cell in which EBV resides. The present disclosure remains consistent with the primary functions of the immune system (defense against pathogenic microbes) and its interactions with metabolic stress.

Changes that take place in the human genome during stress that have prevented researchers from recognizing and treating these virus components effectively. Stress initiates changes to the promyelocytic leukemia nuclear bodies (PML-NB aka ND10) to which several viral components, including human herpes viruses and influenza, have adapted. Virus propagation is restricted by intact PML-NB. However, chemical changes including SUMOylation, disrupt the PML-NB and facilitate the propagation of viral components. Normally, our immune systems recognize the envelope proteins on the viral surface and we generate antibodies to neutralize it (e.g., the basic premise of vaccination). During stress, our bodies can modify these proteins to look entirely different than those of a normal virus (creating neoepitopes) and produce what are termed “exosomes” (sometimes referred to as extracellular vesicles or EVs) that can display these “defective” viral proteins. Moreover, the reactivation of 2 or more viruses by coinfection or superinfection within an individual cell may preclude the ability of an individual virus to form whole virus particles. Human antibodies can recognize these defective viral components or virus-like particles that contain jumbled components from multiple viruses.

In this scenario, when there is a jumbling of components from two or more distinct viruses, the immune response recognizes these modified proteins in combination and reacts differently than when it encounters a routine (single) viral infection, creating cross-reactive (polyspecific) T cells and antibodies. These polyspecific immune components that are produced to target these newly-generated virus particles see the “combination” of the two viruses, rather than a single virus. It is the combination of virus components that appears to “mimic” human proteins (HLA or autoimmune) and cause rejection or autoimmune disease through specific T cells or antibodies.

Most conventional immunosuppressive treatments, to battle organ rejection or autoimmune diseases, target elements of our immune system or metabolic pathways rather than a virus. As a result, these treatments cause side-effects, dampen the immune response and make an individual susceptible to infection.

The disclosed approach allows one to harness these antibodies to diagnose disease and its locale of origin in the human body, and to develop new treatments with them since they target components from the specific cell(s) that contain the specific virus pair(s).

By removing the functional portion of an HLA- or auto-antibody (e.g., the fragment crystallizable (Fc) region) that causes organ or tissue damage, a dye can then be conjugated/attached to the antibody and be introduced at low concentrations back into the body of the diseased patient. This can potentially work like a GPS signal—to locate the exact cells containing the pathogenic virus pair(s) without causing adjacent tissue destruction inherent to the functions of the antibody's Fc region. By geolocating the viruses, they can then be excised or treated at the location of the coinfection. In theory, this will abrogate the need for generalized immunosuppression medications and treatments (which are inconsistent at best and rely on patient compliance) and their accompanying side-effects elsewhere in the body.

Alternatively, these viral antibody specificities can also be incorporated into CAR-T therapies as a substitute (the scFv aka single chain variable fragment) for more non-specific antibody moieties as are currently in use (for example, CD19).

It is envisioned that this will enable virus-infected cells to be killed specifically and keep patients from having to undergo immunoglobulin or immunosuppression therapy for the remainder of their lives.

Another envisioned possibility is to tag nanoparticles with the antibodies. The nanoparticles, conjoined with or “filled” with siRNA or other compounds which can either silence the virus genes from being translated into protein or kill the cell, would be able to geolocate the coinfected cell, be taken in, and perform their specified function.

Another envisioned possibility is to screen small molecule compounds for antagonist reactivity to the specific viral target proteins associated with an individual's HLA-mismatched transplanted organ or autoimmune disease.

Another envisioned utility is improving diagnostic risk assessment. Consistent with the literature, both donor specific antibodies (DSA) and non-DSA may be deleterious and cause transplanted organ rejection. Via the same mechanism (mischaracterized in the past as “idiotype” theory), autoantibodies can cause and exacerbate disease. Unlike other diagnostic tools, the NETRAD algorithm enables the identification of deleterious vs innocuous non-DSA and can also function identically for autoimmune antibodies. By identifying innocuous vs deleterious “idiotype” antibodies, NETRAD provides a more effective tool for physician interpretation of diagnostic HLA assays (e.g., Single antigen bead array, flow cytometry assay) and autoimmune antibody tests to assess the risk of DSA and autoimmune antibodies. This will facilitate physicians to be able to modify testing schedules and determine with greater accuracy and efficacy, the use of desensitization and immunosuppression protocols or innovative treatment options provided by NETRAD. This can facilitate longer graft survival, thereby increasing the organ donor pool, diminish the complications and symptoms of transplant rejection and autoimmunity, and minimize the necessity for using generalized therapeutic treatments that cause side effects.

Based on the inherent characteristics of viral envelope proteins that are included in vaccine formularies, and the adjuvants that are included to stimulate the immune system (e.g., promote the stress-related changes previously referenced), it is recognized that vaccines influence chronic disease processes.

An example of actions for characterizing and isolating anti-viral antibodies to determine cross-reactive patterns with alloantigens (HLA) or autoantigens is described below.

The actions may include utilizing a management system, such as Thermo Fisher Scientific/One Lambda's HISTOTRAC™ full laboratory management system, to process all of the tests, manage patient data, etc.

The actions may first include HLA typing of patient and/or donor according to manufacturer instruction. Any appropriate instrument and/or software may be utilized, including, e.g., Thermo Fisher Scientific/One Lambda's ALLTYPE™ FASTPLEX™ NGS Assay, ION GENESTUDIO™ S5 next-generation sequencing (NGS) system, and TYPESTREAM™ Visual NGS analysis software. As will be understood, based on the instruments, company, any software, the exact procedure will vary.

The actions may then include serum identification and/or characterization of an HLA antibody and titer. Any appropriate instrument and/or software may be utilized, including, e.g., Thermo Fisher Scientific/One Lambda's LABSCREEN™ Single Antigen Beads, LABSCAN3D™ advanced multiplex flow analyzer, and HLA Fusion research Software. As will be understood, based on the instruments, company, any software, the exact procedure will vary. In some embodiments, this may include pre-treating the serum with, e.g., ZEBA™ desalting columns and MELON™ Gel Monoclonal IgG purification support, according to the manufacturer's instructions, to free up undetectable antibodies from high affinity exosomes. In some embodiments, this may further include comparing the specificities of monoclonal anti-HLA antibodies vs patient monospecific sera.

The actions may include performing a viral homology assay.

Performing the assay may include obtaining one or more commercial viral envelope proteins. Various viral envelope proteins that may be appropriate include, e.g., H1 hemagglutinin (Acro Biosystems ctlg #HA1-V52H3), N1 neuraminidase (Acro Biosystems ctlg #NEE-V524k), H3 hemagglutinin (Acro Biosystems ctlg #HA2-V52H3), N2 neuraminidase (Acro Biosystems ctlg #NE2-V5249), Influenza B hemagglutinin (Acro Biosystems ctlg #HAE-V52H3), Influenza B neuraminidase (Acro Biosystems ctlg #NEE-V5245), EBV gp350 (Acro Biosystems ctlg #GPO-E52H6), BKV VP1 (Acro Biosystems ctlg #VP1-A5143), HSV-1 gB (Acro Biosystems ctlg #GHL-H5283), CMV gB (Creative Diagnostics ctlg #DAG-H10041), VZV gB (Sino Biological/FisherScientific ctlg #50-237-1180), and/or SARS-CoV-2 spike (Creative Biomart ctlg #Spike-219VB). Additional envelope proteins may be generated or acquired.

Performing the assay may include obtain HLA/autoantigens. This may be done using commercially technology from, e.g., Thermo Fisher Scientific/One Lambda or Werfen, or by isolating such HLA/autoantigens independently.

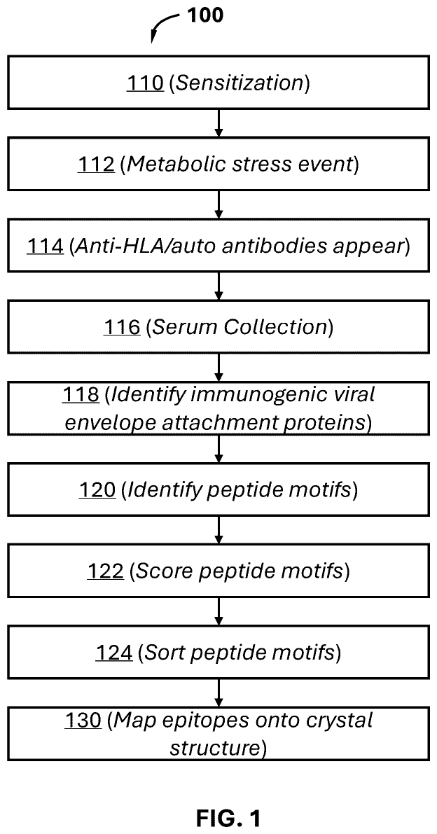

Performing the assay may include utilizing an ELISA assay to detect cross-reactivity. A schematic of a basic cross-reactivity assessment assay can be seen in FIG. 1. Utilizing an ELISA assay may include coating the protein on microtiter plate. See, e.g., Thermo Fisher Scientific's document “Thermo Scientific Immunoassay Plate Guide”, located at assets.thermofisher.com/TFS-Assets/LCD/Scientific-Resources/Immunoassay_Plate_Guide.pdf, the contents of which are incorporated herein by reference. This may include coating a co-infecting viral protein (separately) from, e.g., EBV gp350, EBV gB. This may include coating a plate with specific HLA/autoantigen (separately). This may include coating negative controls (non-specific protein, BSA, PBS-separately).

Utilizing an ELISA assay may include treating coated viral antigen wells positive and negative controls to remove N-glycans.

Utilizing an ELISA may include pre-treatment of tray-coated proteins with, e.g., PNGase F from New England Biolabs.

Utilizing an ELISA assay may include performing one or more tests. This may include incubating corresponding anti-HLA antibody/autoantibody/serum to bind viral envelope protein+/−HLA/autoantigen. This may include incubating non-specific Ab, separately, as negative control. This may then include washing the various incubated products. This may include incubating with secondary antibody (e.g., alkaline phosphatase-labeled goat anti-human IgG (ahIgGAP)). This may then include washing the various incubated products. This may include incubating with a detection reagent such as fluorometric dye (e.g., CDP-STAR™ chemiluminescent dye from, e.g., Thermo Fisher Scientific). This may include detecting/reading/measuring signal emissions such as on a fluorometer (Revvity's ENVISION® XCite 2105 Multimode Plate Reader). This may include analyzing the detected/measured light emissions. In these analyses, it will be expected that viral coated antigens and cross-reactive allo(HLA)/autoantigens would adsorb a specific antibody (Ab) to yield a high signal to noise ratio (e.g., SNR≥5) compared to negative control proteins. These may be compared to NETRAD predictions.

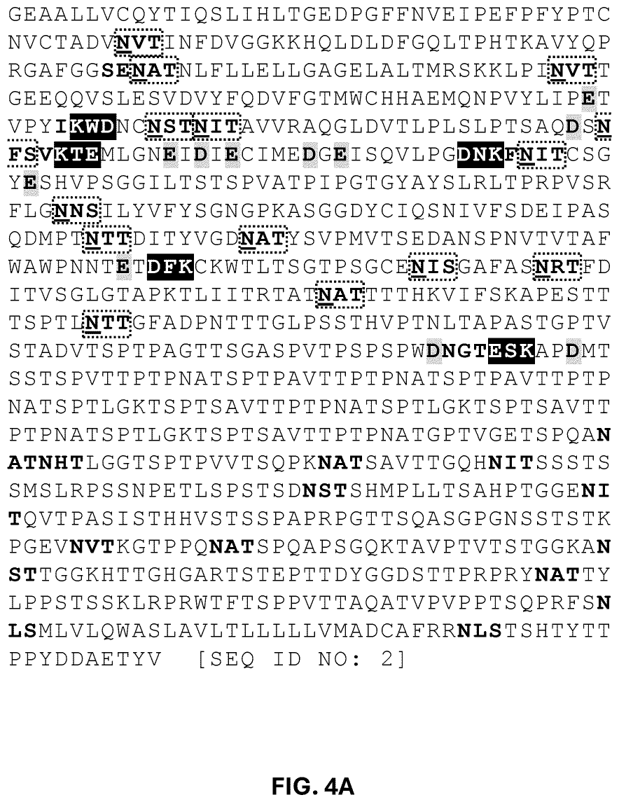

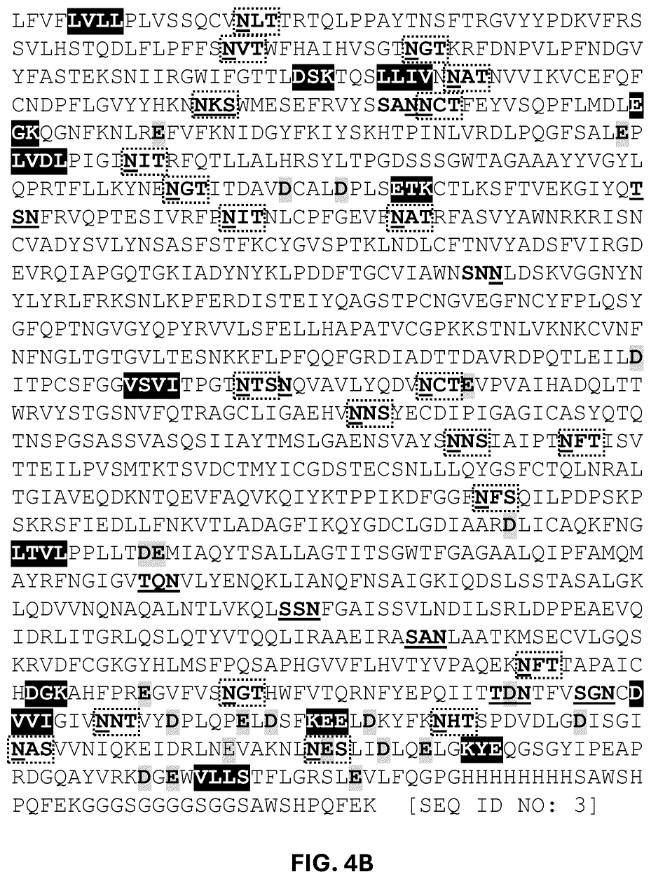

The actions may include isolation and characterizations of exosomes and SUMO (such as SUMO-2) identification. See, e.g., FIGS. 4A and 4B.

This may include acquiring serum with a pre-established threshold level of interfering factors. Such interfering factors may be, e.g., exosomes or virus-like particles, which may be indicated by a high negative control MFI value or low positive control MFI value on LabScreen Single Antigen test.

This may include adsorbing and/or isolating exosomes using alloantibody (e.g., HLA from One Lambda or Immucor)/autoantibody/TLR4/TLR2/exosome biomarkers (e.g., CD9,63,81 from Abcam, ThermoFisher, Creative Biolabs) conjugated to magnetic beads (e.g., LODESTARS™ magnetic beads from Agilent) or column.

This may include using a buffer to lyse exosomes. See, e.g., Subedi P et al. “Comparison of methods to isolate proteins from extracellular vesicles for mass spectrometry-based proteomic analyses”, Anal. Biochem. 2019 Nov. 1:584:113390. doi: 10.1016/j.ab.2019.113390, the contents of which are incorporated by reference herein in its entirety. This may include performing 2-dimensional gel electrophoresis or other technique to purify, isolate, and characterize proteins reacting with a specific allo- or auto-antibody. See, e.g., Chun S et al. “Exosome Proteome of U-87MG Glioblastoma Cells”, Biology (Basel). 2016 Dec. 6; 5(4):50. doi: 10.3390/biology5040050; Ronquist K G et al. “Proteomic analysis of prostate cancer metastasis-derived prostasomes” Anticancer Res. 2010 February; 30(2):285-90; Zubiri I et al. “Kidney tissue proteomics reveals regucalcin downregulation in response to diabetic nephropathy with reflection in urinary exosomes”, Transl Res. 2015 November; 166(5):474-484.e4. doi: 10.1016/j.trsl.2015.05.007, the contents of each of which are incorporated by reference in its entirety.

This may include coating an ELISA plate using allo-/autoreactive primary antibody (based on NETRAD assignment). This may include configuring a sandwich ELISA assay to capture specific protein isolated from exosome and sandwich detection antibody (e.g., anti-SUMO-2-AP conjugate from Abcam, ThermoFisher, Novus Biolog, or Creative Biolabs).

A sandwich ELISA assay is a scientific technique that detects the presence of an antigen or antibody in a sample. It is a solid phase diagnostic method that is widely used to diagnose protozoan and metazoan diseases in humans and animals. As an example: a capture antibody that's highly specific for the antigen may be attached to a solid surface. The antigen to be measured must contain at least two antigenic sites capable of binding to antibody. The sandwich ELISA measures the amount of antigen between two layers of antibodies (capture and detection antibody). A substrate is added, which is changed by the enzyme into a color, electrochemical signal, or fluorescent. The fluorescence or electrochemical signal on the multi-plate well is measured to determine the presence and quantity of antigens.

This sandwich ELISA analysis will demonstrate that the protein captured by the allo/autoAb is specific and is bound to SUMO-2. As is known, a sandwich ELISA measures the amount of antigen between two layers of antibodies (capture and detection antibody). A substrate is added, which is changed by the enzyme into a color, electrochemical signal, or fluorescent. The fluorescence or electrochemical signal on the multi-plate well is measured to determine the presence and quantity of antigens. Alternatively, this may include transferring separated proteins to a gel or membrane and staining w/conjugated allo-/autoantibody for identification.

This may include obtaining amino acid sequence of isolated protein after digesting with SENP3/6/7 enzyme (Millipore SIGMA ctlg #APREST70801; Abcam Ctlg #ab151868) to remove SUMO2/3. See, e.g., Horn D M et al. “Automated de novo sequencing of proteins by tandem high-resolution mass spectrometry”, PNAS. 2000; 97(19): 10313-10317; Alfaro J A et al. “The emerging landscape of single-molecule protein sequencing technologies” Nat Methods. 2021 June; 18(6): 604-617. doi: 10.1038/s41592-β21-01143-1; Wang, M et al. SENP3 regulates the global protein turnover and the Sp1 level via antagonizing SUMO2/3-targeted ubiquination and degradation. Protein Cell. 2016; 7(1):63-77. doi: 10.1007/zl3238-015-β216-7.; Li Y et al. Structural basis for the SUMO2 isoform specificity of SENP7. J Molec Biol. 2022; 434:e167875. This will determine whether protein is an HLA/autoantigen, or is a viral envelope protein or some other component.

The actions may include configuring a diagnostic assay. This may include acquiring viral envelope proteins (which may be commercially available proteins, as disclosed herein). This may include HLA/autoantigens (which may be commercially available, as disclosed herein). This may include coating proteins into individual wells using high pH (e.g., pH≥9.0) coating buffer. Preferably, a multi-well plate array, such as a 96- or 384-well plate, is used. (96-well/384-well plate) using high pH (9.0) coating buffer. This may include QC/validating the specificity and/or sensitivity of the reagents. This may include centrifuging patient whole blood and removing serum and/or plasma. This may include utilizing a chelant, such as an EDTA, to chelate one or more components from the patient's whole blood (such as iron, calcium, etc.). This may include incubating the serum/plasma, and washing. This may include adding and/or incubating anti-human IgG alkaline phosphatase 2° antibody (ahIgGAP) and washing. This may include adding/incubating a fluorometric dye (e.g., CDP-STAR™ chemiluminescent dye as disclosed herein). This may include reading and analyzing a signal to noise ratio using appropriate detection analysis instrumentation and/or software (see Revvity's ENVISION® XCite 2105 Multimode Plate Reader). Cross-reactivity with individual virus envelope proteins may be based on NETRAD and as disclosed herein.

To summarize, configuring an assay for diagnostic use may include isolating a unique viral envelope proteins that have been stripped of N-glycans, using, e.g., PNGase F. It may include coating on microtiter plate, and coating cross-reactive proteins (HLA, autoantigen) and controls at distinct locations. It may include performing a sandwich ELISA using patient sera as source of antibody. It may include determine which viral protein and cross-reactive human protein have a relatively high signal-to-noise ratio. In some embodiments, this may be SNR≥5. In some embodiments, this may be a SNR≥10.

The actions may include using an anti-viral neoepitope antibody (HLA/autoimmune) for locating and/or treating at a disease source.

This may include characterizing one or more specific monoclonal antibody(s) associated with a unique disease. The NETRAD algorithm may be applied to developing antibodies associated with a disease.

This may include removing the Fc region of an antibody or expressing the scFv (aka single chain variable fragment). The F(ab′)2 or scFv can then be conjugated to another molecule, such as, e.g., a dye. The dye may be a dye used for MRI (magnetic resonance imaging), CT (computed tomography), and/or PET (positron emission tomography) scan. Such a conjugated product may allow a scanning device (e.g., MRI, CR, or PET) to locate a cell source within an individual responsible for secreting exosomes that express the viral neoepitopes (and consequent miRNA, DNA, proteins, etc.) associated with the disease. That is, because the conjugated product, having been designed via the NETRAD algorithm, will bind at or near the cell source, the scanning devices can detect the location of the dye and thereby identify a source. Upon locating the viral coinfected cell(s), an appropriate treatment can be selected—localized excision and extraction, or alternatively treatment with small molecules (pharmacologic) or scFab-expressing microvesicles/nanoparticles carrying cytolytic small molecules can be used to cause cytolysis, minimizing the need for more invasive procedures. As will be understood, the disclosed techniques are agnostic with regards to the method of treating the specific disease. Rather, the disclosed technique is intended to aid in the developing of a treatment or in the targeting of a treatment.

The disclosure can be further appreciated in view of the Appendix, which includes four figures (FIGS. 1-4), and the applicant's manuscript entitled “Anti-HLA Antibodies May Be a Subset of Polyreactive Immunoglobulins Generated After Viral Superinfection” (Transplant Immunololgy. 2025 Feb. 13:102197. doi: 10.1016/j.trim.2025.102197, and its supplementary figures and information, each of which is incorporated by reference herein in its entirety.

### Netrad

The NETRAD algorithm is an epitope analysis tool designed to identify viral peptide sequences which share sequence homology with HLA or autoantigens.

The NETRAD model was developed to explore the feasibility of anti-HLA antibodies being generated to target stress-modified, HLA-homologous coinfecting viral components. NETRAD identifies HLA-homologous, gapped viral amino acid sequence motifs (e.g., KAxSxT, where capital letters represent homologous amino acids and “x” represents residues that differ between HLA and viral sequences). It was also considered that viral peptide motifs incorporate post-translational protein modifications (PTM) that might inhibit antibody recognition of specific viral epitopes under normoxic conditions but convert those motifs to antibody-accessible neoepitopes that cross-react with HLA when influenced by cell stressors (e.g., ischemia/reperfusion injury (IRI)). Coinfection and viral persistence, particularly the presence of an EBV component, may be crucial to the generation of these antibodies.

The NETRAD algorithm evolved after observing the emergence of novel anti-HLA antibodies that followed reproducible patterns of exposure to EBV plus a second, coinfecting or superinfecting virus (see Table 1, below), frequently triggered by a stress event. Exposure events included: 1) coinfection by EBV plus a second, distinct virus, 2) superinfection by EBV, preceded by or following infection by a second, distinct virus, or 3) infection by EBV preceded by or following exposure to a second, distinct virus through vaccination.

TABLE 1
Origins of Viral-Derived HLA-Homologous Epitopes.

| Co-Infecting | Virus | Targeted HLA Specificity |
|---|---|---|
| Virus* | Type | (HLA Eplet Registry Designation) |
| Adenovirus | DNA | A*03:01 (65RNA) |
| | | DRB1*11:01(86G) |
| BK Virus | DNA | A*30:01 (70QS, 76VDT, 79GT) |
| | | DQA1*03:02~DQB1*03:02 (2D) |
| | | DQA1*03:02~DQB1*03:02 (76 V) |
| Coronavirus | RNA | A*29:01 (62LQ, 66NV, 80TL) |
| OC43 | | |
| Cytomegalovirus | DNA | A*02:01 (62GE, 74HD) |
| (CMV) | | B*37:01 (44RT, 66IS, 71TD) |
| | | DRB1*03:01 (57DA) |
| EBV | DNA | B*57:01 (44RMA, 62GRN, 80I) |
| Metapneumovirus | RNA | A*11:02 (76VDT) |
| | | A*02:01 (62GE, 107 W) |
| Herpes Simplex | DNA | A*23:01 (65GK) |
| Virus-1 (HSV-1) | | B*51:01 (44RT, 66IF, 76EN) |
| Varicella zoster | DNA | B*55:01 (45EE, 63NI, 80 N, 95 W) |
| | | A*80:01 (76ANT) |
| Human | RNA | DPA1*02:01~DPB1*01:01 |
| | | (83 A, 84DEAV) |
| Rhinovirus | | B*07:02 (44EE, 80 N, 177DK) |
| Human Herpes | DNA | DRB1*15:01 (32YN, 37S, 71 A) |
| Virus-6B | | A*25:01 (65RNA, 76ESI) |
| (HHV-6B) | | |
| Influenza H1N1 | RNA | C*04:01 (14 W, 73AN, 77 N, |
| | | 80 K, 156R) |
| | | DQA1*01:03~DQB1*06:03 |
| | | (3S, 52PQ, 87F) |
| | | DRB1*04:04 (57DA, 70QA, |
| | | 70QT, 86 V, 96Y) |
| Influenza H3N2 | RNA | DRB1*03:01 (16H, 37 N) |
| | | B*13:02 (44MA) |
| | | B*15:12 (76ESN, 163LG) |
| | | B*27:05 (66IC, 131S) |
| Influenza B | RNA | B*44:02, *44:03 (41 T) |
| | | DRB1*07:01 (47YR, 60S) |
| | | DQA1*05:01~DQB1*02:01 |
| | | (56PA, 75S, 76L) |
| SARS-CoV-2 | RNA | C*07:01 (66 N, 80 N) |
| | | DRB1*15:01 (37S, 47F) |

*EBV gp350 or gB is included as a prerequisite to each superinfecting virus pairing.

The timing of exposure to each virus pair more frequently took place in succession (superinfection including EBV or EBV infection preceded by or following vaccination), at times with significant gaps between exposure to EBV and the second, distinct virus. Notably, novel anti-HLA antibodies appeared following exposure to viral components, at which point intact whole virions were either unsuspected (not tested for at the time of alloantibody appearance), undetectable or below the detection threshold according to standard testing methods. These patterns were observed both in the presence and absence of traditional sensitization events (pregnancy, transfused blood products, transplantation). Re-activation of EBV and other viruses by stress has been established. Stressors included oxygen deprivation (e.g., IRI), metabolic imbalance, psychological stress, wounding, toxins, and unrelated infections but were not limited to these events. After identifying patterns of anti-HLA antibodies appearing after viral sensitization (see Table 1), a strategy was developed to identify HLA-homologous viral peptide motifs that might generate polyreactive or cross-reactive HLA-specific epitopes: the NETRAD algorithm. See FIG. 1.

In the NETRAD algorithm (100), a sensitization step (110) may occur, and preferably an EBV infection in combination with an infection from a coinfecting or superinfecting virus. The exact sequence of sensitization may vary; e.g., the infections may be interchangeable or simultaneous. In some instances, the timing of exposure to each virus (e.g., EBV+coinfecting virus) was not necessarily concurrent but often took place in succession prior to the appearance of anti-HLA antibodies.

A metabolic stress event may occur (112), such as hypoxia, ischemia/reperfusion injury [IRI], infection, metabolic changes, exposure to toxins/heavy metals, emotional/psychological stress, etc.

Anti-HLA antibodies may then appear (114). IgM may or may not seroconvert to IgG upon a first encounter, depending upon the metabolic status of the patient and the persistence of viral components in tissue vs. blood.

Serum may then be collected (116) for clinical testing and risk assessment of anti-HLA antibodies.

The algorithm may then include identifying (118) immunogenic viral envelope attachment proteins. For example, by performing in-silico N-lysozyme digestion of EBV gp350/gB+the second virus envelope protein into peptides.

The algorithm may then include identifying (120) all HLA-homologous viral peptide sequence motifs based on the identified attachment proteins. This may be accomplished, e.g., using the Smith-Waterman algorithm. For example, if the EBV peptide is KTQMLGNEIDIECIMEDGEISQVLPGDN (SEQ ID NO: 1), a peptide sequence motif may be identified as DGExxxV.

Consistent patterns of EBV coinfection/superinfection were observed, including either coinfection with a distinct virus or upon exposure to a distinct virus through vaccination, preceding the development of anti-HLA antibodies (see Table 1). The timing of exposure to each virus (EBV+coinfecting virus) was not necessarily concurrent but often took place in succession prior to the appearance of anti-HLA antibodies and frequently followed a stress-related event (hypoxia, ischemia/reperfusion injury [IRI], infection, metabolic changes, exposure to toxins/heavy metals, emotional/psychological stress, etc.).

As noted, identified HLA specificities and viral envelope protein sequences can be obtained from IMGT-HLA or NCBI websites, respectively. Major immunogenic viral envelope attachment proteins are considered the most likely candidates due to their extent of glycosylation and documented immunogenicity.

The algorithm may include scoring (122) each identified HLA-homologous viral peptide sequence motif on HLA crystal structure ectodomain. A truncated table (Table 2) is shown below as an example of identified and scored peptide sequence motifs.

In Table 2, peptides are identified by the Expasy PeptideMass number, followed by an alphabetic indicator of peptide sequence orientation (F=forward, R=reverse). Other columns indicate the virus envelope protein of origin, the number of N-glycan sequons present on the original viral peptide sequence, HLA homology derived from Smith-Waterman analysis of the correlate viral peptide, HLA start residue, an ERP scored location indicating the relative location of the peptide sequence with regard to surface accessibility (0-5), and whether the HLA-correlate peptide sequence is HLA specific (Y/N).

Scoring for antibody accessibility is performed with an ectodornain-relative peptide (ERP) locating system. Peptide locations are scored according to: 1=membrane-distal α-helix; 2=β-pleat membrane-distal side chain; 3=β-pleat membrane-proximal underside; 4 stalk region, located between the β-pleat and membrane proximity; 5=membrane abutment; 0=cryptic locations lacking antibody-accessible residues (e.g., peptide cleft, buried or transmembrane residues). ERP scores of “1-4” are positioned for optimal antibody-binding access within the extracellular space, with a score of “1” being the furthest membrane distal location and having the highest level of antibody accessibility. ERP scores of “0” or “5” were considered cryptic locations, consistent with having been reported to lack clinical relevance.

TABLE 2
Truncated listing of HLA-homologous CMV and EBV peptides derived from
the NETRAD algorithm and sorted to obtain epitope 62GE (HLA-A*02:01).

| | Expasy | | | | | |
|---|---|---|---|---|---|---|
| | generated | | # | | | |
| | peptide/ | Smith Waterman- Derived | N- | | | HLA- |
| Virus | orientation | HLA-A*02:01 Homologous | glycans | | | A*02:01 |
| Protein | F = forward | Gapped (‘x’ or ‘/’) | per | HLA start | ERP | (62GK) |
| Origin | R = reverse | Viral Peptide Sequences | peptide | residue | score | specific |
| CMV | 2591.3683R | AxGxVD | 1 | 24 | 0 | No |
| gB | 887.4846F | GY | 0 | 26 | 0 | No |
| | 2606.2555R | xxxxFVx/SxRxE | 1 | 29 | 1 | Yes |
| | 4128.0879R | xxVRFDxx | 0 | 32 | 0 | No |
| EBV | 4062.0236R | VxFxSDxAS | 3 | 34 | 1 | Yes |
| gp350 | | | | | | |
| CMV | 1064.4378F | DSD | 0 | 37 | 2 | No |
| gB | 1064.4378R | DSD | 0 | 37 | 2 | No |
| | 1140.6663F | xxPxxPxx | 0 | 45 | 2 | No |
| | 5631.6528F | ExRA/xQxGxxxxDGxT | 1 | 46 | 2 | Yes |
| EBV | 843.457R | xxxEGPx | 1 | 52 | 1 | Yes |
| gp350 | | | | | | |
| CMV | 3492.6124F | xWxxxxRxxxxxxx | 1 | 59 | 1 | No |
| gB | | | | | | |
| EBV | 3091.4268F | DGExxxV | 0 | 61 | 1 | Yes |
| gp350 | | | | | | |
| CMV | 9306.7576F | DxxT(9*x)Hxx/xxxTLRGYxNxxxA | 3 | 61 | 1 | Yes |
| gB | 1085.6214F | RxxxAHx | 0 | 65 | 1 | Yes |
| | 974.5451R | RxVK | 0 | 65 | 1 | No |
| | 8111.8549F | SxTxR(6*x)RxxYxQ/SEAxSH | 3 | 71 | 1 | Yes |
| | 1085.6214R | THxV | 0 | 73 | 1 | Yes |
| EBV | 1749.8918F | HxxDL | 0 | 74 | 0 | Yes |
| gp350 | | | | | | |
| CMV | 1417.8498F | HR | 0 | 74 | 1 | Yes |
| gB | 1304.6514R | DLG | 0 | 77 | 2 | Yes |
| | 1152.5895R | xxGxxxGY | 1 | 77 | 1 | Yes |
| | 1876.9076F | YNQx | 1 | 85 | 1 | No |

For information about the epitope derivation of 62GE (HLA-A*02:01), see Table 3, below).

The algorithm may include sorting (124) the peptide motifs by HLA start residue (see, e.g., Tables 2 and 3, where, for example, the “DGExxxV” motif has an HLA start residue of 61. The truncated table shown as Table 3, below, is sorted by the HLA start residue, from lowest to highest.

TABLE 3
Epitope derivation of HLA-A*02:01 (62GE) from CMV and EBV.

| | Expasy | | | Smith-Waterman- | | |
|---|---|---|---|---|---|---|
| | PeptideMass | Virus | # N- | generated | HLA | # Ab |
| Antibody | generated | Envelope | glycans/ | gapped peptide | start | accessible |
| specificity | peptide ID# | Protein | peptide | sequence | residue | residues |
| HLA- | 56316528F | CMV gB | 1 | ExRA--- | 46 | 12 |
| A*02:01 | | | | xQxGxxxxDGxT | | |
| (62GE) | 843457R | EBV | 1 | xxxEGPx | 52 | |
| | | gp350 | | | | |
| | 34926124F | CMV gB | 1 | xWxxxxRxxxxxxx | 59 | |
| | 30914268F | EBV | 0 | DGExxxV | 61 | |
| | | gp350 | | | | |
| | 10856214F | CMV gB | 0 | RxxxAHx | 65 | |
| | 10856214R | | | THxV | 73 | |
| | 9745451R | | 0 | RxVK | 65 | |

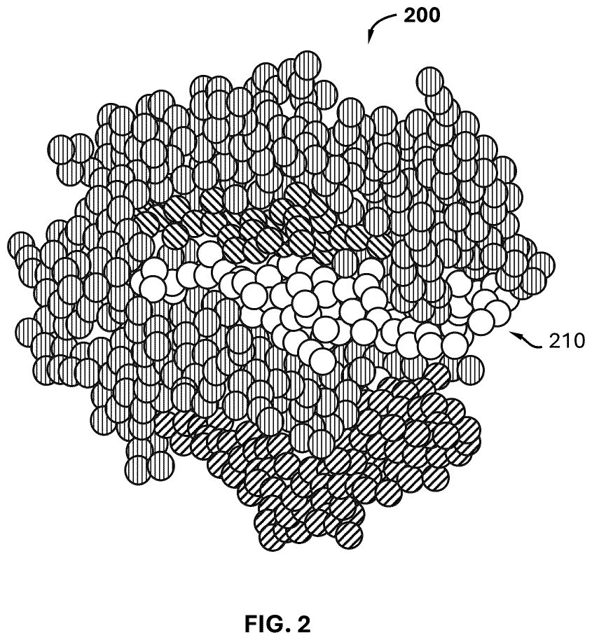

The algorithm may include mapping (130) HLA-specific epitopes from viral peptides superimposed onto a crystal structure, such as a pHLA3D crystal structure (see FIG. 2). In FIG. 2, the epitope (210) is shown as white-filled circles superimposed onto a crystal structure (200) for the epitope of Table 3.

The mapping may include various requirements. For example, 6 viral peptide motifs should encompass the HLA-specific eplet (“functional eplet”). 7 or more (such as 7-25) residues should lie within a 15-angstrom radius of the functional eplet. The epitope may not be impeded by an HLA cleft-presented peptide. At least one peptide of the 6 viral peptides encompassing the functional eplet must be derived from EBV. At least one peptide must be derived from the second virus. At least one peptide of the 6 viral peptides encompassing the functional eplet should harbor an N-glycan sequon (NxS/T).

The epitope should be accessible to a DSA. There may be multiple epitopes present from the same virus pair.

### Example

HLA and corresponding viral protein sequences were acquired from IMGT-HLA or NCBI. Immunogenic viral envelope attachment proteins were considered likely candidates due to the extent of their glycosylation. In silico N-lysozyme-digested viral peptide libraries were created utilizing EXPASY PeptideMass.

Peptides may be listed and identified by mass. N-glycan motifs may be identified (bold, italicized, and underlined in example for EBV gp350 (see SEQ ID NO: 2), below:

### Expasy Peptide Mass Output:

| Mass | position | peptide sequence |
|---|---|---|
| 9670.5676 | 644-740 | KNATSAVTTGQHNITSSSTSSMSLRPSSNPET |
| | | LSPSTSDNSTSHMPLLTSAHPTGGENITQVTP |
| | | ASISTHHVSTSSPAPRPGTTSQASGPGNSSTS |
| | | T |
| | | [SEQ ID NO: 12] |
| 9061.7248 | 827-907 | KLRPRWTFTSPPVTTAQATVPVPPTSQPRFSN |
| | | LSMLVLQWASLAVLTLLLLLVMADCAFRRNLS |
| | | TSHTYTTPPYDDAETYV |
| | | [SEQ ID NO: 13] |
| 7810.4894 | 291-362 | KASGGDYCIQSNIVFSDEIPASQDMPTNTTDI |
| | | TYVGDNATYSVPMVTSEDANSPNVTVTAFWAW |
| | | PNNTETDF |
| | | [SEQ ID NO: 14] |
| 7679.6430 | 423-501 | KAPESTTTSPTLNTTGFADPNTTTGLPSSTHV |
| | | PTNLTAPASTGPTVSTADVTSPTPAGTTSGAS |
| | | PVTPSPSPWDNGTES |
| | | [SEQ ID NO: 15] |
| 6778.4402 | 227-290 | KFNITCSGYESHVPSGGILTSTSPVATPIPGT |
| | | GYAYSLRLTPRPVSRFLGNNSILYVFYSGNGP |
| | | [SEQ ID NO: 16] |
| 6151.9151 | 1-56 | MEAALLVCQYTIQSLIHLTGEDPGFFNVEIPE |
| | | FPFYPTCNVCTADVNVTINFDVGG |
| | | [SEQ ID NO: 17] |
| 5894.8430 | 110-160 | KLPINVTTGEEQQVSLESVDVYFQDVFGTMWC |
| | | HHAEMQNPVYLIPETVPYI |
| | | [SEQ ID NO: 18] |
| 5130.5310 | 502-554 | KAPDMTSSTSPVTTPTPNATSPTPAVTTPTPN |
| | | ATSPTPAVTTPTPNATSPTLG |
| | | [SEQ ID NO: 19] |
| 4558.2543 | 597-643 | KTSPTSAVTTPTPNATGPTVGETSPQANATNH |
| | | TLGGTSPTPVVTSQP |
| | | [SEQ ID NO: 20] |
| 4367.0559 | 786-826 | KHTTGHGARTSTEPTTDYGGDSTTPRPRYNAT |
| | | TYLPPSTSS |
| | | [SEQ ID NO: 21] |
| 4062.0236 | 161-198 | KWDNCNSTNITAVVRAQGLDVTLPLSLPTSAQ |
| | | DSNFSV |
| | | [SEQ ID NO: 22] |
| 3794.9897 | 73-108 | KAVYQPRGAFGGSENATNLFLLELLGAGELAL |
| | | TMRS |
| | | [SEQ ID NO: 23] |
| 3643.7696 | 365-400 | KWTLTSGTPSGCENISGAFASNRTFDITVSGL |
| | | GTAP |
| | | [SEQ ID NO: 24] |
| 3092.4108 | 199-226 | KTEMLGNEIDIECIMEDGEISQVLPGDN |
| | | [SEQ ID NO: 25] |
| 2029.0448 | 555-575 | KTSPTSAVTTPTPNATSPTLG |
| | | [SEQ ID NO: 26] |
| 2029.0448 | 576-596 | KTSPTSAVTTPTPNATSPTLG |
| | | [SEQ ID NO: 26] |
| 1844.0236 | 401-417 | KTLIITRTATNATTTTH |
| | | [SEQ ID NO: 27] |
| 1749.8918 | 58-72 | KHQLDLDFGQLTPHT |
| | | [SEQ ID NO: 28] |
| 1665.8191 | 749-765 | KGTPPQNATSPQAPSGQ |
| | | [SEQ ID NO: 29] |
| 1118.6051 | 766-777 | KTAVPTVTSTGG |
| | | [SEQ ID NO: 30] |
| 843.4570 | 741-748 | KPGEVNVT |
| | | [SEQ ID NO: 31] |
| 735.3631 | 778-785 | KANSTTGG |
| | | [SEQ ID NO: 32] |

Along with proteolysis at a specific amino acid residue (except for the 1^{st} peptide from each sequence, which begins with Methionine in most cases, all other digested peptides began with lysine (K)), N-Lysozyme also removes N-glycans from proteins and is upregulated during hypoxia. N-glycans (bold, underlined, and italicized above) were recognized by the amino acid sequence motif NxS/T/C, where x is any amino acid except proline (i.e., EBV gp350 peptides above). Also, any motif with proline appearing directly adjacent to the N-glycan sequon had to be excluded (i.e., NP-T, PNxT or NxSP-). It was predicted that N-glycans are removed from the viral envelope protein via N-lysozyme during hypoxia to facilitate glycolysis, thereby unmasking new epitopes (neoepitopes).

In silico digested N-Lysozyme-generated viral peptides were assessed for 1) HLA homology and specificity by utilizing the Smith-Waterman algorithm and for 2) antibody accessibility to clinically relevant HLA binding sites.

Peptides are assessed for homology by Smith-Waterman in forward and reverse directions, independently. After obtaining the homology assessment for each peptide via the Emboss Water web tool, each of the HLA peptides are listed in an MS Excel spreadsheet (Dryad repository). HLA homology is listed using standard amino acid nomenclature and can be found at the IMGT-HLA website. Gapped amino acid positions are listed as “x” or spacers “-”. “x” indicates an identical number of gaps when comparing HLA vs viral sequences whereas “-” indicates a gap that is offset between the HLA and corresponding viral sequence.

For example: EBV peptide 30914268F sequence=KANSTTGG in “forward” direction. Notably, the underlined N-glycan sequon (NST) in the peptide above would be expected to block antibody binding to this sequence until the N-glycan is removed by enzymatic degradation (N-lysozyme) or attrition to be used for the glycolytic cycle to generate ATP. The corresponding gapped sequence homology to the HLA-A locus protein ( . . . KAHSQTD . . . ), was determined by the Smith Waterman algorithm as KAxSxT (HLA-A locus residue 68-73). A complete listing of viral peptides and corresponding HLA homologies can be found at the Dryad repository.

Peptides are identified by the Expasy PeptideMass number in the first column, followed by an alphabetic indicator of peptide sequence orientation (F=forward, R=reverse). Unique spreadsheet columns indicate the virus envelope protein of origin, the number of N-glycan sequons present on the original viral peptide sequence (not shown above, see figure below), HLA homology derived from Smith-Waterman analysis of the correlate viral peptide, HLA start residue, an ERP scored location (see below) indicating the relative location of the peptide sequence with regard to surface accessibility (0-5), and whether the HLA-correlate peptide sequence is HLA specific (Y/N). Additional columns may appear for reference but were unused in tabulating results.

A comprehensive listing of the immunogenic viral envelope protein sequences and individual peptide sequences, derived from Expasy PeptideMass in-silico N-lysozyme digestion of viral envelope proteins, are included in the MS Word doc (Dryad repository). All peptide identification numbers/peptide mass (except for those </=5 amino acids in length) are included in a corresponding tab in the MS Excel spreadsheet (CompleteNetradHLAPeptideSortingData; Dryad).

Once homology is generated for all viral peptides, each peptide can be mapped to HLA-specific amino acid residues on the respective HLA crystal structure. HLA crystal structures were obtained from pHLA3D (www.phla3d.com.br) or the Protein Database (Research Collaboratory for Structural Bioinformatics Protein Data Bank. HLA epitopes derived from viral homology were assessed, for antibody accessibility to published epitopes, utilizing pHLA3D or 1D-3D view at PDB.

In some instances, HLA crystal structures were acquired from the PDB database and pHLA3D, YASARA, or the PDB “Explore in 3D” software was used to superimpose homologous viral peptide motifs onto the corresponding HLA crystal structure and evaluate each motif for antibody accessibility.

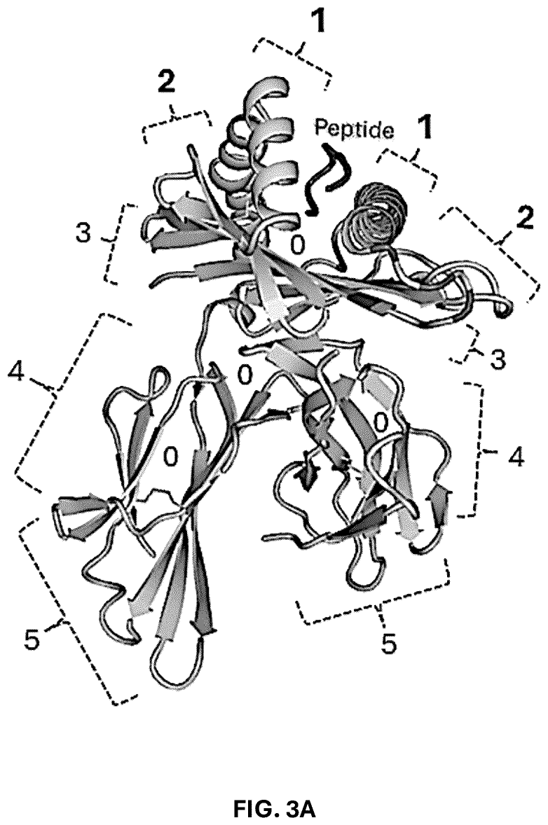

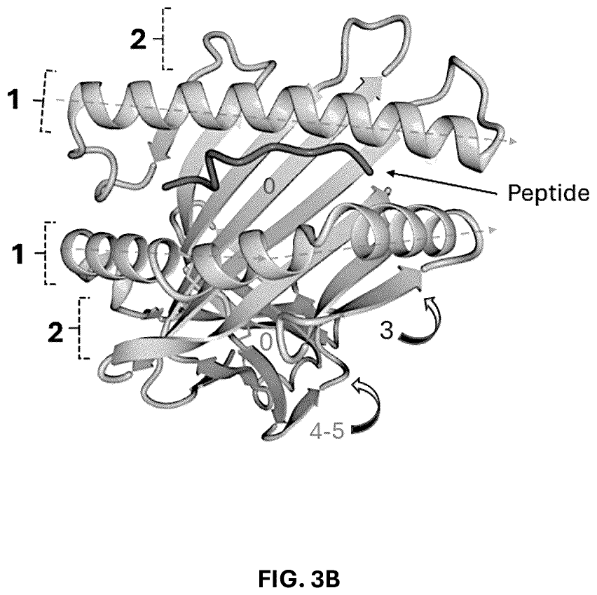

Peptide locations were scored (by an Ectodomain Relative Peptide scoring system or ERP), according to FIGS. 3A and 3B, as follows: 1=membrane-distal α-helix; 2=β-pleat membrane-distal side chain; 3=β-pleat membrane-proximal underside; 4=stalk region, located between the β-pleat and membrane proximity; 5=membrane abutment; 0=cryptic locations lacking antibody-accessible residues (e.g., peptide cleft, buried or transmembrane residues). ERP scores of 1-4 are positioned for optimal antibody-binding access within the extracellular space, with a score of 1 being the furthest membrane distal location and having the highest level of antibody accessibility. ERP scores of 0 or 5 were considered cryptic locations, consistent with having been reported to lack clinical relevance.

Scored HLA-homologous viral peptide motifs were then sorted in a spreadsheet according to IMGT-HLA cross-referenced amino acid sequence position. Each peptide was assessed for its ability to confer HLA specificity based on the presence of distinguishing amino acids at hypervariable sequence positions (yes/no). Peptide motifs were then sorted by ERP score and the peptide amino-terminal residue (according to its sequence position relative to the corresponding mature HLA protein, i.e., HLA start residue in Tables 2 and 3).

Next, combinations of viral peptides (ERP score=1-4) were mapped onto crystal structures to determine whether a clinically relevant HLA-specific epitope could be demarcated to encompass the functional eplet, and if so, the minimum number of peptides necessary to do this.

HLA-epitopes derived from viral peptide combinations are determined to be clinically relevant only when an epitope is comprised of between 7-25 antibody-accessible amino acids (ERP score=1-4), included residues of the functional eplet to convey HLA specificity, and is encompassed within an established 15-angstrom (Å) radius. Importantly, HLA epitopes demarcated by viral peptides must be unimpeded by the naturally presented peptide held within the HLA cleft. This ensured that HLA cleft-presented peptides would not subject the epitope to continuous amino acid residue variation.

Rudimentary physical parameters were established by which conformational differences between homologous HLA and virus components might influence the binding of an anti-HLA antibody. The inter-residue differential (measured in Å) was sampled for thirty-seven residue pairs from twenty-eight different peptides (13%) on eleven different viruses, based on crystal structure availability. Considering that a viral peptide rests in a relatively linear conformation within the confines of an HLA protein cleft during antigen presentation, the differential distance between amino acid pairs as they appeared on the correlate virus or HLA proteins was calculated. This was done in effort to determine whether an anti-HLA antibody would be flexible enough to bridge conformational changes imparted by the tertiary structure of either protein, while remaining rigid enough to be classified as a polyreactive antibody. Comparable residues that were antibody-accessible on a specific HLA epitope were assessed with the correlate residues of the virus protein. No distinction was made between HLA Class I vs HLA Class II. It was considered that differentials <4.9-6.3 Å could be accommodated by an antibody's paratope, while differentials >10 Å may not. Differences in the number of gapped spaces (‘x’) between homologous residues for distinct peptides (e.g., 2 ‘x’s between serine and threonine vs 4 ‘x’s between alanine and threonine for peptide motif “KAxSxxT”) were accounted for, and it was determined whether the inter-residue differential met statistical significance across the selected population of peptides (one-way ANOVA analysis using GraphPad PRISM v10.2.2). In addition, the predicted surface contact diameter for each virus-derived HLA epitope were compared at 2 distinct measurement points, including the maximum diameter, against the standard fifteen A radius of an antibody-antigen interface.

PTM sequons (attachment sites comprised of specific amino acids), specifically for Small Ubiquitin-like Molecules (SUMO) and N-glycans that might influence the generation of neoepitopes under cell stress conditions (e.g., IRI) were identified independently for each viral envelope protein (examples in FIGS. 4A and 4B) and each viral peptide sequence, respectively (examples in Tables 2-3, with a comprehensive listing at the Dryad data repository). Predicted SUMO binding sites were identified by the consensus sequon motif Kx(E/D) (where “x” is any amino acid), or an alternative SUMO interacting motif (e.g., LLIV, VSVI, LTVL, etc.), accompanied by requisite flanking acidic residues (aspartic acid=D/glutamic acid=E). N-glycan sequons were identified by consensus sequon motifs NxS/T/C, where “x” is any amino acid except proline. Any N-glycan motif with proline appearing directly adjacent to the N-glycan sequon was excluded (e.g., NT, NxT or NxS).

For each epitope analysis, the amino acid sequence of the HLA allele representing the antibody specificity (higher affinity or strongest reacting allele in the circumstance of cross-reactive antibody) was aligned with and compared to the HLA typing of a patient, or patient and donor pair. Amino acid residues from identified viral peptide homology were superimposed (e.g., highlighted) onto the antibody-specific allele sequence. These homologous residues were then assessed for 1) their ability to impute antibody specificity by complementing the sequence of the antibody-specific allele, 2) ability to exclude self and donor alleles, not targeted by the antibody, via repelling residues, and 3) the number of antibody-specific residues accessible at the epitope-paratope interface. In all cases, the residue(s) of the functional eplet were integral to meeting these criteria. All viral-derived HLA epitopes were compared to standardized eplets located at the HLA Eplet Registry. Antibody specificities that have historically been considered false positive or weakly autoreactive (e.g, HLA-A*11:02, HLA-B*15:12, HLA-DRB1*04:04) were also sampled to determine whether a correlated epitope could be demarcated using the algorithm.

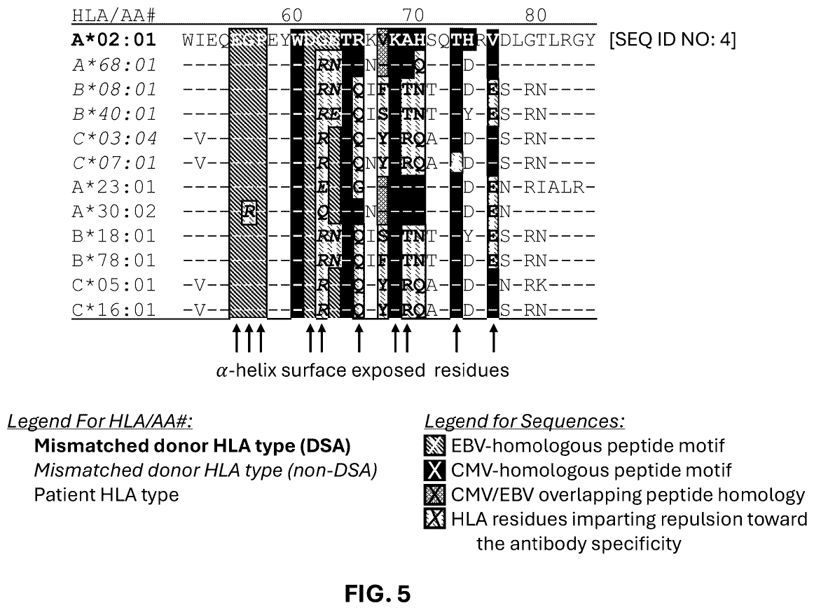

The NETRAD algorithm identified 215 peptides, from eighteen distinct viral envelope proteins encompassing fourteen unique DNA or RNA viruses, which exhibited semi-linear or gapped homology with HLA epitopes. Seventy-two distinct antibody-accessible HLA-specific epitopes were demarcated, each from a combination of six peptide motifs, including at least one peptide from EBV (gp350 or gB) and at least one peptide from a distinct immunogenic viral envelope protein. Tables 2-3 and FIG. 5 are provided as an example to demonstrate the NETRAD derivation of an HLA epitope (62GE), its respective crystal structure and the corresponding immunogenic viral envelope proteins and peptides from which it was derived with respect to the HLA typing of a patient/donor pair. Included among the tables are elements of the viral peptide scoring system, peptide orientation, the number of N-glycan sequons present on each viral peptide and the corresponding HLA start residue for each homologous viral peptide subsequence. Viral peptide-derived HLA epitopes display an average of fifteen antibody-accessible residues (range=9-27), consistent with the number of amino acids necessary to attain specificity and encompass the functional eplet and epitope-to-paratope interface. An average of five N-glycan PTM sequons (range=2-11) are distributed among each combination of six viral peptides. Additionally, each identified viral protein contains a minimum of two motifs predicting covalent and/or non-covalent binding to SUMO-2/3 (FIGS. 4A-4B). By superimposing viral peptide motifs (Table 3) onto patient and mismatched donor HLA sequences, we demonstrate that homology from cytomegalovirus (CMV) and EBV can demarcate epitope 62GE specificity (HLA-A*02:01) (FIG. 5). The crystal structure detail corresponding to this antibody-accessible HLA epitope is presented as red-colored spheres (Table 3).

Thus, in some instances, NETRAD attempts to identify and map an HLA specific epitope, where six viral peptide motifs encompass the HLA-specific eplet (at least one of the six must be derived from EBV, and at least one from a second (e.g., coinfecting/superinfecting) virus, and at least 1 of the 6 harbors an N-glycan sequon), 7-25 residues lie within a 15 Å radius of the functional (HLA-specific) eplet, where the epitope cannot be impeded by HLA cleft-presented peptide. NETRAD also attempts to determine if the epitope is accessible to DSA and whether multiple epitopes are present form the same virus pair.

To ascertain whether the NETRAD algorithm was specific, it was determined whether virus peptide combinations used to derive a given HLA specificity could demarcate additional HLA epitopes. Although additional epitopes were discovered using this strategy (Table 1), a total of 137 HLA specificities could not be demarcated using this method. Virus envelope proteins that had not been identified with viral sensitization were also randomly paired, for their capacity to map peptides to HLA-specific epitopes and generate an anti-HLA antibody. Neither potential HLA/viral pair correlation (HLA-A*30:01[epitope 70QS]≠EBV gp350+HSV-1 gB or HLA-B*27:05[epitope 71ATD]≠EBV gp350+H3N2 hemagglutinin/neuraminidase) yielded peptide combinations that met the criteria to demarcate the respective epitope specificity. In total, 139 viral pairs served as negative controls for HLA antibody specificities.

Upon comparing the measurement differential for 37 distinct homologous residue pairs on the correlate HLA or virus protein, 54% exhibited a differential <3.0 Å, 81% <4.9 Å and 100% <8.0 Å. ANOVA analysis, accounting for both the A-differential and the number of gaps between homologous residues, indicated a statistically significant correlation for the differential among the population of residue pairs tested (p>0.0001). In addition, the average predicted surface contact diameter of all virus-derived epitopes was congruent to the standard 15 Å radius of an antibody-antigen binding interface (x̅_{diameter}=13.2 Å; range=6.5-19.5 Å; comparing all 62 epitopes, t-test p value <0.0001).

Training and validation of all seventy-two epitope analyses yielded consistent HLA specificity with the corresponding standardized eplets at the HLA Eplet Registry (Table 1). Surprisingly, eight of the seventy-two viral-derived HLA epitopes appeared to duplicate HLA Eplet Registry specificities but were demarcated by peptides from distinct superinfecting virus pairs (Table 1), inferring that a near identical HLA epitope specificity can be generated from distinct viral entities. On the other hand, only one viral pair (EBV+H1N1) appeared to generate a duplicate epitope specificity (156R) from distinct peptide combinations.

Preliminary in silico results suggest that NETRAD demonstrates the capacity for complete demarcation of HLA-specific epitopes via identification of HLA-homologous viral peptide motifs and their superimposition onto HLA crystal structures.

The NETRAD algorithm was developed as the basis for a diagnostic tool with the ability to elucidate complete HLA-specific epitopes from superinfecting viral components. NETRAD was conceived in an effort to resolve the problems associated with characterizing anti-HLA antibodies. Because of the anomalies associated with current anti-HLA antibody testing, we considered that these antibodies could exhibit lower affinity to a single [viral] target yet have higher avidity by targeting multiple pathogenic components resulting from coinfection or superinfection, while exhibiting the capacity to cross-react with HLA.

The evolution of NETRAD necessitated the consideration of metabolic stressors that not only can influence the modification of viral antigenic determinants, but also the inflammatory response to these determinants.

Part of the value of NETRAD lies in its ability to facilitate more accurate and comprehensive HLA epitope analyses and risk assessment of DSA, including the discernment of innocuous versus injurious non-DSA that may influence allograft rejection (see Table 1 and explanation below). NETRAD provides a logical reason why HLA Class II antibodies may be more deleterious than HLA Class I antibodies (see below) and can also explain the development of autoreactive HLA antibodies. These distinguishing characteristics give NETRAD the potential to increase the predictive value of anti-HLA antibody identification and provide more comprehensive risk assessment for allograft rejection.

Using NETRAD, it has been consistently found that a combination of six viral peptides can demarcate a single HLA epitope. Each combination of six peptides exhibited an average of five N-glycan PTM motifs (range=2-11; see Tables 2 and 3 as an example). N-glycans are known to alter the T cell repertoire, obstruct epitope recognition, and inhibit antibody binding. During cell stress (e.g., IRI), an increase in glycolysis is necessary to maintain the demand for cellular ATP and may competitively scavenge resources that prevent the ligation of N-glycans to viral antigens. To compound the process, slower enzymatic digestion of immune complexes is facilitated by the FcRn in the acidic lysosomal compartment, enabling N-glycan cleavage by N-lysozyme. The possibility that viral proteins could also be modified by N-lysozyme-bearing exosomes cannot be ruled out. Either of these metabolic changes infer a mechanism conducive to neoepitope formation that can expose masked antigenic determinants prior to antigen presentation.

SUMOylation consensus sequons were also apparent on each identified viral envelope protein (see FIGS. 4A-4B). Based on the sequence location of SUMO-2/3 sequons, it was predicted that naturally accessible viral epitopes (under normoxic conditions) may be masked by SUMO-2/3 ligation under cell stress conditions. SUMO-2/3 are markedly upregulated during oxidative stress and IRI, and as such have been incorporated into the algorithm because of their integral association with traditional sensitization events (IRI accompanies transplantation, transfusion and pregnancy). Under the conditions of cell stress, SUMO2/3 and N-glycan modification could create morphologically and antigenically distinct viral envelope proteins, exhibiting neoepitopes (the “NE” of NETRAD). Natural polyreactive antibodies are known to bind antigenically altered cells. It is plausible that the combination of coinfecting or superinfecting virus components, altered by PTM, would likely incur stearic changes that preclude the formation of whole virions while facilitating the expression of modified viral proteins on the outer surface of exosomes, defective virus-like particles (VLPs), or apoptotic membrane debris (terms used interchangeably with “soluble HLA antigen” from here on for the purpose of context).

An integral step to NETRAD, the Smith-Waterman algorithm used in this example was limited by its inability to identify more than one gapped subsequence of amino acid residues within a single peptide. This limitation prompted the inventor to identify peptide homology in forward and reverse directions (Tables 2-3). Unlike enzymatic processes which require directionality to be translated, presentation of an antigenic peptide from the HLA cleft to a T-cell receptor can take place whether the peptide is oriented in either forward or reverse direction. As a result, a surpassing number of HLA Class II NETRAD-derived epitopes exhibited >6 homologous motifs, amid a combination of six peptides, compared to HLA Class I (88% vs 36%, respectively). This may reflect a higher HLA Class II antibody avidity and potentially account for the association of HLA Class II antibodies with poorer graft survival.

While current transplant sensitization theory implicates exposure to mismatched HLA as a prerequisite to developing anti-HLA antibodies, DSA being of particular concern for allograft rejection, the NETRAD algorithm demonstrates evidence that anti-HLA antibodies may cross-react with neoepitopes from superinfecting viral components, lending it the capacity to identify whether combinations of DSA and non-DSA may be deleterious to graft survival or result in tolerance. According to NETRAD, the ability of anti-HLA antibodies to neutralize PTM-modified superinfecting viral components (currently recognized as “soluble HLA antigen” but which also may have been mischaracterized as anti-idiotype antibody) can explain the actively acquired tolerance of a transplanted organ in the presence of DSA.

For example, via competitive binding, polyreactive antibody may neutralize relatively higher affinity viral components (i.e., “soluble HLA antigen”) that mimic HLA-A*02:01 (DSA) before binding to a mismatched organ typed as HLA-A*02:01, thereby facilitating graft tolerance. In contrast, the deleterious effects of DSA, manifesting as both acute or chronic AMR, can be explained by either of two mechanisms that revolve around the neutralization of modified viral components: 1) the presence of DSA in the absence of “soluble HLA antigen,” or 2) the simultaneous appearance of DSA and “soluble HLA antigen” plus what has been characterized as anti-anti-idiotype antibody (which also can be polyreactive).

For example (refer to Table 1), the appearance of anti-HLA-B*37:01 as a non-DSA, along with anti-HLA-A*02:01 DSA may exacerbate rejection. In this scenario, both antibodies target the same viral components (EBV gp350 and CMV gB) yet may compete to neutralize these virus-like particles that contain HLA-homologous epitopes (i.e., VLP=“soluble HLA antigen”). While anti-HLA-A*02:01 DSA alone would preferentially bind the VLP, an increasing titer of anti-HLA-B*37:01 would increase the relative concentration of free DSA (anti-HLA-A*02:01) to bind the mismatched allograft antigen (HLA-A*02:01) and cause AMR. In the case where anti-HLA-A*02:01 antibody stabilizes with a decline in the concentration of such VLPs, allograft rejection would also be expected to ensue. As such, the equilibrium between VLPs, DSA and non-DSA (i.e., HLA-B*37:01=anti-anti-idiotype antibody) will determine the severity of AMR. Idiotype antibodies were originally discovered through indirect testing methods.

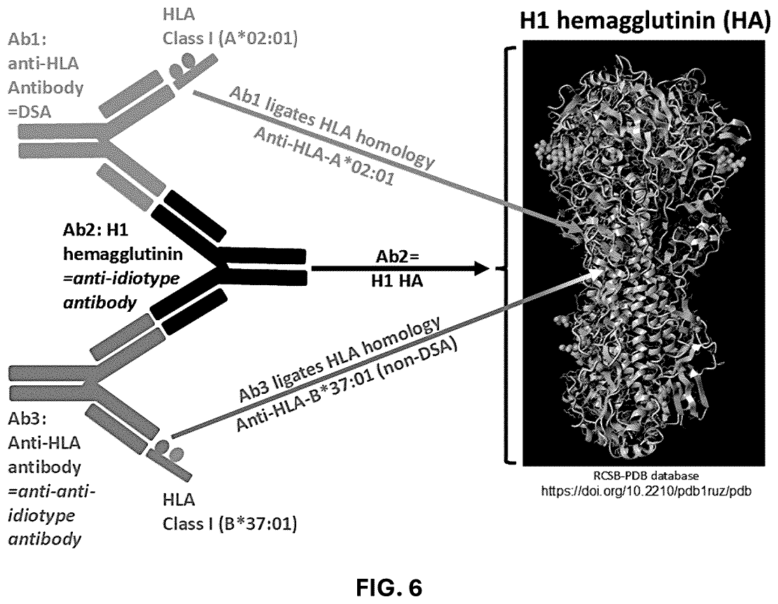

Consequently, anti-anti-idiotype antibodies could not be distinguished from antibodies targeting a competitive viral epitope situated adjacent to an HLA-homologous and cross-reactive viral epitope. See FIG. 6. In FIG. 6, the idiotype model infers that anti-idiotype antibody (Ab2) can be generated to neutralize the variable chain specific region F(ab′)2 of an anti-HLA antibody (Ab1), and also that anti-anti-idiotype antibody (Ab3) can be generated to neutralize the F(ab′)2 of Ab2. This assumes that the Ab3 paratope targets a distinct epitope on Ab2 than the paratope of Ab2 which is ligated to the F(ab′)2 of Ab1- a highly improbable proposition that was based on indirect testing methods. According to the immunologically feasible NETRAD model (right) which was designed after historical precedence, the viral envelope protein behaves identically to anti-idiotype antibody Ab2 and ligates both Ab1 and Ab3, whose targeted epitopes (blue and white circled regions) lie immediately adjacent to one another on the viral envelope protein H1 HA. Stearic hindrance would preclude the concurrent binding of both Ab1 and Ab3, thereby exacerbating symptoms of AMR when Ab1=DSA, as previously described. H1 HA, neutralized by Ab1 DSA in the absence of Ab3 can facilitate graft tolerance. Crystal structure of Influenza A virus (Right: A/South Carolina/1/1918(H1N1) hemagglutinin) PDB 1RUZ contains homology to HLA Class I epitopes 65RNA, 73AN, 77N, 80K and Class II epitope 52PQ. HLA homology is shielded by N-glycans under normal physiologic conditions.

The feasibility of this scenario can be predicted by NETRAD's capacity to determine both DSA and non-DSA epitopes from the same virus protein pairs listed in Table 1. Non-DSA has been reported to adversely influence AMR prognosis. NETRAD provides an explanation for this by predicting that the ratio of HLA-homologous viral components (“anti-idiotypes”) to DSA titer and the presence or absence of “anti-anti-idiotype antibodies” may determine tolerance versus the onset and severity of AMR. In a pre-transplant scenario, the organ allocation system may choose to avoid transplanting a candidate that has developed antibody targeting HLA-B*37:01 under the premise that it may inevitably develop anti-HLA-A*02:01 DSA. On the other hand, in the post-transplant scenario, the appearance of anti-HLA-B*37:01 would be a caveat alerting the transplant team to the potential of developing DSA (to HLA-A*02:01) and thereby warrant modification of the testing schedule and possibly the patient's immunosuppressive regimen. These capabilities distinguish NETRAD from other bioinformatics tools.

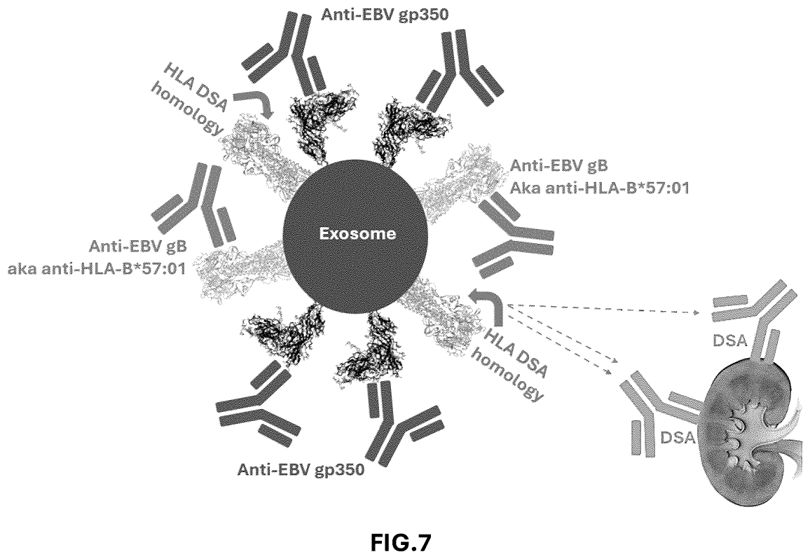

A coinfected or superinfected individual cell could potentially express two or more PTM-modified viral envelope proteins on an exosome or VLP, including EBV gp350 plus gB as well as the corresponding envelope protein from the superinfecting virus. Antibody specificity HLA-B*57:01 has previously been shown to be present in 40% transplant patients. According to observations in Table 1 that are supported by NETRAD analysis, anti-HLA-B*57:01 (epitope 62GRN) indicates the presence of PTM-modified EBV gB. This antibody specificity could behave like a competitive anti-anti-idiotype antibody among any transplant recipient for whom HLA-B*57:01 is not a mismatched antigen (non-DSA). Similar to the previously mentioned example, the concurrent appearance of anti-HLA-B*57:01 antibody with DSA can also be predicted to exacerbate AMR by increasing the relative concentration of DSA. See FIG. 7. In FIG. 7, anti-HLA-B*57:01 (epitope 62GRN) can behave like an anti-anti-idiotype non-DSA antibody by neutralizing an exosome expressing modified viral components that exhibit neoepitopes. A DSA antibody, that could bind to gp350 or a distinct virus envelope antigen (gray hemagglutinin exhibiting correlate HLA-DSA homology) and a transplanted organ, would more likely bind to the transplanted organ when the titer of the 62GRN specificity competes with the DSA to neutralize the exosome/virus-like particle. This would exacerbate AMR.

The HLA-homologous gapped viral peptide motifs identified by NETRAD were conserved and have withstood antigenic drift and shift (refer to Table 4, below); individual virus strains used for comprehensive analyses can be found in the Dryad data repository.

TABLE 4
Truncated sequence alignment of hemagglutinins
(HA) exhibiting HLA homology.

| ID | Sequence |
|---|---|
| 2022_A_New | KAILVVMLYTFTTANADTLCIGYHANNSTDT |
| York_04 | VDTVLE |
| | [SEQ ID NO: 5] |
| 2009_A_New | KAILVVLLYTFATANADTLCIGYHANNSTDT |
| York_1682 | VDTVLE |
| | [SEQ ID NO: 6] |
| 1995_A_New | KAKLLVLLCAFTATYADTICIGYHANNSTDT |
| York_650 | VDTVLE |
| | [SEQ ID NO: 7] |
| 1977_A_USSR_90 | KAKLLVLLCALSATDADTICIGYHANNSTDT |
| | VDTVLE |
| | [SEQ ID NO: 8] |
| 1968_A_Wisconsin_1 | KAILLVLLCTFAATNADTLCIGYHANNSTDT |
| | VDTVLE |
| | [SEQ ID NO: 9] |
| 1934_A_Puerto | KANLLVLLCALAAADADTICIGYHANNSTDT |
| Rico_8 | VDTVLE |
| | [SEQ ID NO: 10] |
| 1918_A_New_York_1 | EARLLVLLCAFAATNADTICIGYHANNSTDT |
| | VDTVLE |
| | [SEQ ID NO: 11] |

Table 4 shows Truncated protein sequences from distinct H1N1 virus strains, listed by year, demonstrate the semi-conserved nature of gapped HLA-homologous viral peptide residues (shown bolded, italicized, and underlined) over the past 106 years. Refer to Table 1 and doi for antibody specificity HLA-C*04:01, peptide 40000259F, and corresponding homologous residues for this motif (AxADxxxxGYxNxSxDxxxT. The Smith-Waterman algorithm accounts for the sequence gap between H1 HA and HLA-C*04:01 (tyrosine [24Y] and asparagine [27N]) and recognizes contiguous homology within the peptide. Except for the transitory mutation of H1 HA residue fifteen, which mutates between threonine (T: 1918/1968/1977/1995) and alanine (A: 1934/2009/2022), the gapped peptide 40000259F motif has remained conserved. H1 HA residues 17, 18, 23, 24, 27, 29, 31 correlate with HLA antibody-accessible residues 73, 74, 83, 84, 86, 88, 90 (see Dryad data repository, epitope 80K). H1 HA residues 15 (A/T) and 35 (T) correlate with antibody-inaccessible HLA residues 71 and 94.

Coinfection or superinfection plus stress could provide the necessary precondition for the generation of these peptides and explain why polyreactive antibodies may exhibit relatively low binding affinity to their viral targets (in comparison to a monoclonal antibody targeting a single viral epitope) while cross-reacting with HLA. It is suspected that due to the scrambling of mixed viral components at the time of antigen presentation, viral components transferred from a donated organ (or potentially originating from the patient) may therefore account for the generation of anti-HLA antibodies. In support of this, reactivated EBV components can be transported via B cells or exosomes throughout the body. Stress-mediated reactivation and modification of viral components can explain why DSA develops months to years after transplanting an organ.

Thus, a novel mechanism is disclosed, involving viral superinfection that has the potential to drive the production of Class I and II anti-HLA antibodies that can cause both acute and chronic antibody-mediated allograft rejection. Implementation of the NETRAD algorithm revealed 215 HLA-homologous viral peptide motifs from fourteen distinct virus pairs to enable the demarcation of seventy-two different HLA-specific epitopes. In addition, the identification of 139 HLA epitopes that could not be demarcated by specific virus pairs served as negative controls. It was discovered that EBV major envelope glycoproteins gp350 and gB, together or in conjunction with distinct coinfecting or superinfecting viral envelope components, may explain the production of anti-HLA antibodies and provide an explanation for why antibodies targeting HLA Class II proteins tend to be more deleterious compared to those targeting HLA Class I.

Donor-specific anti-HLA antibodies (DSA), generated from allosensitization-associated events, can result in AMR and reduce the survival time of transplanted organs. Globally, organ transplant teams depend upon HLA compatibility testing and early identification of DSA to ensure optimal graft survival. It is expected that anti-HLA antibodies can be tested for polyreactivity to the viral components identified herein, under experimental conditions conducive to an IRI environment.

NETRAD prompts a reevaluation of the mechanism of transplant rejection, leading to some challenging implications for diagnostic testing, allograft rejection treatments and vaccine administration among immunocompromised patients. The NETRAD model is an innovative epitope analysis strategy with the potential to increase the predictive value of anti-HLA antibody assay interpretation. NETRAD was developed to facilitate increased allograft survival and organ availability. NETRAD may enable improvements to donor selection, facilitate more accurate interpretation and risk assessment of diagnostic tests used to assess donor compatibility, and predict the onset and severity of AMR. NETRAD may also provide new directions for more efficacious and patient-specific treatment strategies, above those currently employed for the treatment of AMR.

What is claimed is:

1. A method for developing a diagnostic interpretation tool and antibody-based treatment, comprising:
identifying coinfecting or superinfecting viral agents in organ transplant or autoimmune patients, prophylactically (pre-transplant or pre-disease) that are at risk of developing rejection (alloimmune/non-self in the case of transplantation; autoimmune/self in the case of autoimmune disease) or post-disease onset.
identifying the concurrence of deleterious human leukocyte antigen (HLA) donor-specific antibodies (DSA) (HLA DSA) or autoantibodies with non-DSA or anti-anti-idiotype antibodies or exosomes containing modified viral components to determine the exacerbation of allograft rejection and/or autoimmune disease;
receiving a target HLA- or auto-antibody, where the target HLA- or auto-antibody has been identified by:
obtaining a plurality of HLA, autoantigen, and viral protein sequences;
creating at least one in silico library of a plurality of enzyme-digested viral peptides;
assessing each enzyme-digested viral peptide for:
HLA or autoantigen homology and specificity; and
antibody accessibility to one or more clinically relevant HLA or autoantigen binding sites; and
assigning a relative surface-accessibility score to HLA or autoantigen viral peptide motifs depending on their location relative to an ectodomain, specifically for accessibility to antibody binding;
forming a modified antibody by removing a functional portion of target HLA- or auto-antibody;
forming a target antibody by attaching one or more functional molecules to the modified antibody.

2. The method of claim 1, wherein the HLA, autoantigen, and viral protein sequences are obtained from one or more databases.

3. The method of claim 1, wherein each enzyme-digested viral peptide is an N-lysozyme digested viral peptide.

4. The method of claim 1, wherein assessing each enzyme-digested viral peptide for HLA or autoantigen homology and specificity includes utilizing the Smith-Waterman algorithm with bidirectionality.

5. The method of claim 1, further comprising mapping at least six enzyme-digested viral peptides that impute gapped homology to HLA-specific or autoantigen-specific amino acid residues on a respective crystal structure, wherein a minimum of one and maximum of five of the six enzyme-digested viral peptides must be derived from Epstein-Barr virus (EBV) envelope glycoprotein gp350.

6. The method of claim 5, further comprising obtaining HLA or autoantigen crystal structures from one or more databases and assessing the HLA or autoantigen crystal structures for antibody accessibility to an epitope, where the identifying of homologous peptide sequences shared between viral envelope proteins and HLA antigens or autoantigens comprises viral-derived HLA or autoimmune epitopes displaying at least 7 antibody-accessible residues.

7. The method of claim 1, further comprising identifying on viral envelope protein sequences and individual peptide sequences, independently, a post translational modification (PTM) that is capable of influencing generation of a neoepitope.

8. The method of claim 7, wherein the neoepitope is determined by the presence of at least one N-glycan within individual peptide sequences and at least one small ubiquitin-like modifier (SUMO) within the viral envelope protein.

9. The method of claim 1, further comprising determining a motif to be a clinically relevant HLA or autoantigen binding site, for HLA only when the motif is unimpeded by peptides presented in an HLA cleft.

10. The method of claim 1, further comprising mapping a surface-accessible HLA-homologous or autoantigen-homologous viral peptide to a respective HLA or autoantigen crystal structure to determine whether an antibody specificity, comprised of at least 7 amino acids including a functional eplet, could be encompassed inside a 15-angstrom radius.

11. The method of claim 1, wherein the functional portion of the HLA- or auto-antibody includes the fragment crystallizable (Fc) region.

12. The method of claim 1, wherein the functional molecule includes a dye that is conjugated or attached to the modified antibody.

13. The method of claim 12, further comprising introducing a plurality of target antibodies into a subject and allowing the target antibodies to bind to a cell containing a pathogenic virus pair.

14. The method of claim 13, further comprising excising or treating cells at a location of the target antibodies bound to the pathogenic virus pair in the subject.

15. The method of claim 1, wherein the functional molecule is a chimeric antigen receptor (CAR) that includes a transmembrane domain and a signaling domain.

16. The method of claim 15, wherein the CAR single chain variable fragment (scFv) incorporates the identified polyspecific anti-microbial antibody.

17. The method of claim 1, wherein the functional molecule is a nanoparticle that comprises one or more small interfering RNA (siRNA) molecules.

18. The method of claim 1, where the identifying of homologous peptide sequences shared between viral envelope proteins and HLA antigens or autoantigens comprises identifying at least one coinfection of a cell by two distinct viruses.

19. A method for estimating the risk for antibody-mediated rejection of cells, tissue or organs based on information comprising a transplant donor's and a transplant recipient's comprising:
determining donor- and recipient-exposures to HLA-homologous viral components resulting from prior exposures to viral infections, bacterial infections, blood transfusion, prior transplantations, pregnancies, metabolic stress, or vaccinations, that may function as an HLA sensitization event;
determining the likelihood of cross-reactivity and antibody mediated rejection to occur if transplantation were to proceed, comprising:
combining viral envelope protein sequences into a viral peptide library;
creating at least one in silico library of a plurality of enzyme-digested viral peptides, generating viral peptide sub-sequences;
assessing each enzyme-digested viral peptide sub-sequence for:
HLA- or autoantigen-homology and -specificity; and
antibody accessibility to one or more clinically relevant HLA or autoantigen binding sites;
scoring the viral peptide sub-sequences by assigning a relative surface-accessibility score to HLA or autoantigen viral peptide motifs depending on their location relative to an ectodomain, specifically for accessibility to antibody binding;
assessing the location of each motif of the corresponding HLA crystal structure to determine α-helix surface-accessibility;
determining motifs unimpeded by peptides presented in the HLA cleft;
mapping surface-accessible, HLA-homologous viral peptides to respective HLA crystal structures;
identifying post-translational modifications, specifically N-glycan and SUMO sequons, that may influence the generation of neoepitopes on viral envelope protein sequences and on individual peptide sequences;
identifying homologous peptide sequences shared between viral envelope proteins and HLA antigens or autoantigens;
estimating the risk for antibody-mediated rejection of a transplant donor's cells, tissue or organs in a transplant recipient's body.

20. The method of claim 19, further comprising
obtaining protein sequences for a donor's and a transplant recipient's HLA-type;
obtaining a plurality of HLA, autoantigen, and viral envelope protein sequences known to be involved in a patient's prior exposures to viral infections, bacterial infections, blood transfusion, prior transplantations, pregnancies, metabolic stress, or vaccinations.


SEQUENCE LISTING (32 sequences)

SEQ ID NO: 1 - protein, 28 aa, Homo sapiens
KTQMLGNEIDIECIMEDGEISQVLPGDN

SEQ ID NO: 2 - protein, 907 aa, Homo sapiens
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 3 - protein, 1286 aa, Homo sapiens
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 4 - protein, 34 aa, Homo sapiens
WIEQEGPEYWDGETRKVKAHSQTHRVDLGTLRGY

SEQ ID NO: 5 - protein, 37 aa, Homo sapiens
KAILVVMLYTFTTANADTLCIGYHANNSTDTVDTVLE

SEQ ID NO: 6 - protein, 37 aa, Homo sapiens
KAILVVLLYTFATANADTLCIGYHANNSTDTVDTVLE

SEQ ID NO: 7 - protein, 37 aa, Homo sapiens
KAKLLVLLCAFTATYADTICIGYHANNSTDTVDTVLE

SEQ ID NO: 8 - protein, 37 aa, Homo sapiens
KAKLLVLLCALSATDADTICIGYHANNSTDTVDTVLE

SEQ ID NO: 9 - protein, 37 aa, Homo sapiens
KAILLVLLCTFAATNADTLCIGYHANNSTDTVDTVLE

SEQ ID NO: 10 - protein, 37 aa, Homo sapiens
KANLLVLLCALAAADADTICIGYHANNSTDTVDTVLE

SEQ ID NO: 11 - protein, 37 aa, Homo sapiens
EARLLVLLCAFAATNADTICIGYHANNSTDTVDTVLE

SEQ ID NO: 12 - protein, 97 aa, Homo sapiens
KNATSAVTTGQHNITSSSTSSMSLRPSSNPETLSPSTSDNSTSHMPLLTSAHPTGGENITQVTPASISTHHVSTSSPAPRPGTTSQASGPGNSSTST

SEQ ID NO: 13 - protein, 81 aa, Homo sapiens
KLRPRWTFTSPPVTTAQATVPVPPTSQPRFSNLSMLVLQWASLAVLTLLLLLVMADCAFRRNLSTSHTYTTPPYDDAETYV

SEQ ID NO: 14 - protein, 72 aa, Homo sapiens
KASGGDYCIQSNIVFSDEIPASQDMPTNTTDITYVGDNATYSVPMVTSEDANSPNVTVTAFWAWPNNTETDF

SEQ ID NO: 15 - protein, 79 aa, Homo sapiens
KAPESTTTSPTLNTTGFADPNTTTGLPSSTHVPTNLTAPASTGPTVSTADVTSPTPAGTTSGASPVTPSPSPWDNGTES

SEQ ID NO: 16 - protein, 64 aa, Homo sapiens
KFNITCSGYESHVPSGGILTSTSPVATPIPGTGYAYSLRLTPRPVSRFLGNNSILYVFYSGNGP

SEQ ID NO: 17 - protein, 56 aa, Homo sapiens
MEAALLVCQYTIQSLIHLTGEDPGFFNVEIPEFPFYPTCNVCTADVNVTINFDVGG

SEQ ID NO: 18 - protein, 51 aa, Homo sapiens
KLPINVTTGEEQQVSLESVDVYFQDVFGTMWCHHAEMQNPVYLIPETVPYI

SEQ ID NO: 19 - protein, 53 aa, Homo sapiens
KAPDMTSSTSPVTTPTPNATSPTPAVTTPTPNATSPTPAVTTPTPNATSPTLG

SEQ ID NO: 20 - protein, 47 aa, Homo sapiens
KTSPTSAVTTPTPNATGPTVGETSPQANATNHTLGGTSPTPVVTSQP

SEQ ID NO: 21 - protein, 41 aa, Homo sapiens
KHTTGHGARTSTEPTTDYGGDSTTPRPRYNATTYLPPSTSS

SEQ ID NO: 22 - protein, 38 aa, Homo sapiens
KWDNCNSTNITAVVRAQGLDVTLPLSLPTSAQDSNFSV

SEQ ID NO: 23 - protein, 36 aa, Homo sapiens
KAVYQPRGAFGGSENATNLFLLELLGAGELALTMRS

SEQ ID NO: 24 - protein, 36 aa, Homo sapiens
KWTLTSGTPSGCENISGAFASNRTFDITVSGLGTAP

SEQ ID NO: 25 - protein, 28 aa, Homo sapiens
KTEMLGNEIDIECIMEDGEISQVLPGDN

SEQ ID NO: 26 - protein, 21 aa, Homo sapiens
KTSPTSAVTTPTPNATSPTLG

SEQ ID NO: 27 - protein, 17 aa, Homo sapiens
KTLIITRTATNATTTTH

SEQ ID NO: 28 - protein, 15 aa, Homo sapiens
KHQLDLDFGQLTPHT

SEQ ID NO: 29 - protein, 17 aa, Homo sapiens
KGTPPQNATSPQAPSGQ

SEQ ID NO: 30 - protein, 12 aa, Homo sapiens
KTAVPTVTSTGG

SEQ ID NO: 31 - protein, 8 aa, Homo sapiens
KPGEVNVT

SEQ ID NO: 32 - protein, 8 aa, Homo sapiens
KANSTTGG